(19) United States
(12) Patent Application Publication
(10) Pub. No.: US 2022/0143005 A1
(43) Pub. Date: May 12, 2022

(54) NAPHTHALIMIDE DERIVATIVES AS ANTI-PARASITIC AGENTS FOR THE TREATMENT OF LEISHMANIASIS AS WELL AS VIRAL, BACTERIAL AND NEOPLASTIC DISEASES

(71) Applicants: ISTANBUL MEDIPOL UNIVERSITESI, Istanbul (TR); YEDITEPE ÜNIVERSITESI, Istanbul (TR)

(72) Inventors: Mustafa GUZEL, Istanbul (TR); Mehmet Hikmet ÜÇISIK, Istanbul (TR); Fikrettin SAHIN, Istanbul (TR); Elif KESKIN, Istanbul (TR); Zeynep ISLEK, Istanbul (TR)

(21) Appl. No.: 17/417,143

(22) PCT Filed: Dec. 24, 2019
(86) PCT No.: PCT/TR2019/051195

§ 371 Date: Jun. 22, 2021

(30) Foreign Application Priority Data
Dec. 27, 2018 (TR) 2018/20740

(51) Int. Cl. A61K 31/473; A61K 45/06; A61P 33/02; C07D 217/24

(52) U.S. Cl. CPC A61K 31/473; C07D 217/24; A61P 33/02; A61K 45/06

(57) ABSTRACT

Disclosed are naphthalimide derivatives and in particular e.g. N-aryl-substituted naphthalimidopropylamine derivatives (i.e. 2-[3-(amino)propyl]-1H-benz[de]isoquinoline-1,3(2H)-dione derivatives) such as e.g. such as e.g. (Formula I) or (Formula III) as anti-parasitic agents for the treatment of Leishmaniasis. The compounds could also be useful to treat viral, bacterial and/or neoplastic diseases. The description discloses exemplary synthesis as well as biological tests against Leishmania infantum parasites (e.g. pages 54 to 58; examples 1 to 7). Exemplary compounds are: (example 3)(example 4) 2-(3-((5-amino naphthalene-1-yl)amino)propyl)-1H-benzo[de]isoquinoline-1, 3(2H)-dione (example 5) 2-(3-((5-amino naphthalene-1-yl)amino)propyl)-1H-benzo[de]isoquinoline-1,3(2H)-dione (example 6).

The present invention is related to bisnaphthalimidopropyl (BNIP) derived unique drug molecules, to the usage of said unique drug molecules to treat viral, bacterial and neoplastic diseases and the preparation of a pharmaceutical composition from said unique molecules that are suitable to be used in treating viral, bacterial and neoplastic diseases.

### PRIOR ART

Bacterial, viral and neoplastic diseases affect a majority of the world's population and they play a crucial role in the determination of parameters such as average lifespan, life quality, etc., of people. These types of people lead to significant losses in labor power and economical burdens due to treatment expenses.

In the prior art, agents are available to treat bacterial, viral, parasitic and neoplastic diseases, however, unique agents are still required which shall provide effective treatment.

As there are not a lot of drugs in the prior art, as the application methods require medical supervision, and they cause severe side effects, makes the treatment of these diseases difficult, and this situation necessitates the development of novel active agents.

As a result of the studies carried out, it has been found that naphthalimide compounds such as mitonaphid were effective on uterine cancer and leukemia, however in the clinical studies that were carried out it has been noted that when the dose was increased these agents led to the toxicity of the central nervous system.

When the prior art is taken into consideration, the invention owners aim to develop a molecule that is suitable to be used in treating viral, parasitic, neoplastic diseases that eliminate the negative aspects of known molecules such as naphthalimide derived molecules.

Another aim of the invention is to develop new molecules that do not have dose restricting toxicity problems, that are highly soluble and bioavailable contrary to the known molecules.

In accordance with these aims the invention owners who have carried out studies, have developed molecules that are suitable for treating bacterial, viral, neoplastic diseases that are shown with Formula I, Formula II, Formula III, Formula IV, Formula V, Formula VI, Formula VII, Formula VIII.

### DETAILED DESCRIPTION OF THE INVENTION

The present invention is related to molecules illustrated with Formula I.

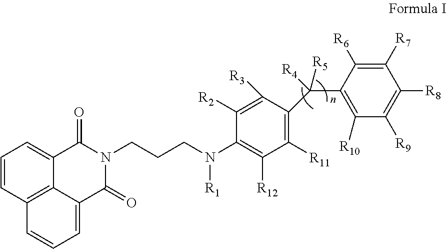

wherein
-n is a natural number between 0 to 12;
R₁, R₂, R₃, R₄, R₅, R₆, R₇, R₈, R₉, R₁₀, R₁₁, and R₁₂ are independently selected from the group of;
—H, —NH₂, —NHR_{X}, —NR_{X}R_{Y}, —NHNR_{X}, —N(R_{X})(NHR_{Y}), —N(R_{X})(NR_{Y}R_{Z}), R_{X}, R_{Y}, R_{Z}, Cl, F, Br, I, —OH, —NO, —NO₂, —COOH, —OR_{X}, —SO₂R_{X}, —SO₂NHR_{X}, —SO₂NR_{X}R_{Y}, —CN, —COOR_{X}, —CONHR_{X}, —CONR_{X}R_{Y}, —NHCOCH₂R_{X}, —NHCOCHR_{X}R_{Y}, —NHCNHR_{X}, —NHCOR_{X}, —NHCONHR_{X}, —NHSO₂R_{X} and;
R_{X}, R_{Y}, and R_{Z} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

The term “alkane” as used within the scope of the invention, defines straight or branched saturated hydrocarbon chains.

The term “alkene” as used within the scope of the invention, defines straight or branched hydrocarbon chains that comprise at least a carbon-carbon double bond.

The term “alkyne” as used within the scope of the invention, defines straight or branched hydrocarbon chains that comprise at least a carbon-carbon triple bond.

According to a preferred embodiment of the invention the molecules according to the invention are illustrated with Formula I;

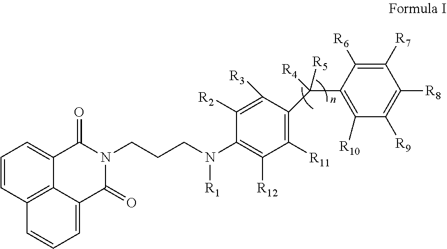

n takes the value of 0, or 1 or 2 or 3 or 4 or 5 or 6 or 7 or 8 or 9 or 10 or 11 or 12;
R₁, R₂, R₃, R₄, R₅, R₆, R₇, R₈, R₉, R₁₀, R₁₁ and R₁₂ are independently selected from the group of;
—H, —NH₂, —CH₃, —CH₂CH₃, —CH₂CH₂CH₃, —CH₂CH₂CH₂CH₃, —CH₂CH₂CH₂CH₂CH₃, —CH₂CH₂CH₂CH₂CH₂CH₃, —CH₂CH₂CH₂CH₂CH₂CH₂CH₃, —CH₂CH₂CH₂CH₂CH₂CH₂CH₂CH₃, —CH₂CH₂CH₂CH₂CH₂CH₂CH₂CH₂CH₃, CH₂CH₂CH₂CH₂CH₂CH₂CH₂CH₂CH₂CH₃, —CH₂CH₂CH₂CH₂CH₂CH₂CH₂CH₂CH₂CH₂CH₃, —CH₂CH₂CH₂CH₂CH₂CH₂CH₂CH₂CH₂CH₂CH₂CH₃, —C(CH₃)₃, Cl, F, Br, I, —OH, —OCH₃, —OCH₂CH₃, —OCH₂CH₂CH₃, —OCH₂CH₂CH₂CH₃, —OCH₂CH₂CH₂CH₂CH₃, —OCH₂CH₂CH₂CH₂CH₂CH₃, —NHCH₃, —NHCH₂CH₃, —NHCH₂CH₂CH₃, —NHCH₂CH₂CH₂CH₃, —NHCH₂CH₂CH₂CH₂CH₃, —NHCH₂CH₂CH₂CH₂CH₂CH₃, —N(CH₃)₂, —N(CH₂CH₃)(CH₃), —N(CH₂CH₃)₂, —N(CH₂CH₃)(CH₂CH₂CH₃), —N(CH₃)(CH₂CH₂CH₃), N(CH₂CH₂CH₃)₂, —NHNH₂, —NHNHCH₃, —NHNHCH₂CH₃, —NHNHCH₂CH₂CH₃, —NHN(CH₃)₂, —NHN(CH₂CH₃)₂, —NHN(CH₃)(CH₂CH₃), —N(CH₃)(NHCH₃), —N(CH₃)(N(CH₃)₂), —COOH, —SO₂CH₃, —SO₂CH₂CH₂, —SO₂CH₂CH₂CH₃, —SO₂CH₂CH₂CH₂CH₃, —SO₂CH₂CH₂CH₂CH₂CH₃, —SO₂CH₂CH₂CH₂CH₂CH₂CH₃, —CN, —CONH₂, —CONHCH₃, —CONHCH₂CH₃, —CONHCH₂CH₂CH₃, —CONHCH₂CH₂CH₂CH₃, —CONHCH₂CH₂CH₂CH₂CH₃, —CONHCH₂CH₂CH₂CH₂CH₂CH₃, —CON(CH₃)₂, —CON(CH₃)(CH₂CH₃), —CON(CH₂CH₃)₂, —CON(CH₂CH₃)(CH₂CH₂CH₃), —CON(CH₃)(CH₂CH₂CH₃), —CON(CH₂CH₂CH₃)₂, —COCH₃, —COCH₂CH₃, —COCH₂CH₂CH₃, —COCH₂CH₂CH₂CH₃, —COCH₂CH₂CH₂CH₂CH₃, —COCH₂CH₂CH₂CH₂CH₂CH₃, —COOCH₃, —COOCH₂CH₃, —COOCH₂CH₂CH₃, —COOCH₂CH₂CH₂CH₃, —COOCH₂CH₂CH₂CH₂CH₃, —COOCH₂CH₂CH₂CH₂CH₂CH₃, —COCH(CH₃)₂, —COC(CH₃)₃, —COCH(CH₂CH₃)₂, —COCH(CH₃)(CH₂CH₃), —COCH₂CH₂CH₃, —COCH₂CH₂CH₂CH₃, —COCH₂CH₂CH₂CH₂CH₃, —COCH₂CH₂CH₂CH₂CH₂CH₃, —SO₂NH₂, SO₂NHCH₃, SO₂NHCH₂CH₃, —SO₂NHCH₂CH₂CH₃, SO₂N(CH₃)₂, —SO₂N(CH₃)(CH₂CH₃), —SO₂N(CH₂CH₃)₂, —SO₂N(CH₃)(CH₂CH₂CH₃), —SO₂N(CH₂CH₂CH₃)₂, —NHCONHCH₃, —NHCON(CH₃)₂, —NHCONHCH₂CH₃, —NHCON(CH₃)(CH₂CH₃), —NHCON(CH₂CH₃)₂, —NHCOCH(CH₃)(NH₂), —NHCOCH(CH₃)₂, —NHCOCH(CH₃)(NHCH₃), —NHCOCH(CH₃)(N(CH₃)₂), —NHCOCH(CH₂CH₃)₂, —NHCOCH(CH₂OH)(NH₂), —NHCOCH(CH₂CH₂OH)(NH₂), —NHCOCH(CH₂CH₂OH)(NHCH₃), —NHCOCH(CH₂OH)(NHCH₃), NHCOCH(CH₂OH)(N(CH₃)₂), NHCOCH(CH₂OH)(N(CH₂CH₃)₂), —NHCOCH(CH₂SH)(NH₂), —NHCOCH(CH₂CH₂SH)(NH₂), —NHCOCH(CH₂CH₂CH₂SH)(NH₂), —NHCOCH(CH₂SH)(NHCH₃), —NHCOCH(CH₂SH)(NHCH₂CH₃), —NHCOCH(CH₂SH)(N(CH₃)₂),

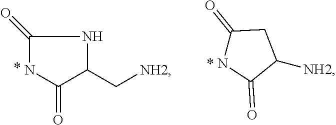 [SMILES: NCC1NC(=O)NC1=O ; NC1CC(=O)NC1=O]

—NH(CNH)NH₂, —NH(CN(CH₃))NH₂, —NH(CN(CH₂CH₃))NH₂, —NH(CN(CH₃))NH(CH₃), —NH(CN(CH₃))N(CH₃)₂, —NH(CNH)NHCH₃, NH(CNH)N(CH₃)₂, —NH(CO)NHCH₂CH₂NH₂, —NH(CO)NHCH₂NH₂, —NH(CO)NHCH₂CH₂NHCH₃, —NH(CO)N(CH₃)(CH₂CH₂NH₂), —NH(CO)CH₂CH₂NH₂, —NH(CO)CH₂NH₂, —NH(CO)CH₂CHCH₂NH₂, —NH(CO)CH₂CH₂CH₂CH₂NH₂, —NH(CO)CH₂CH₂NHCH₃, —NH(CO)CH₂CH₂N(CH₃)₂, —NH(CO)CH₂CH₂NH(CH₂CH₃), —NH(CO)CH₂CH₂N(CH₃)(CH₂CH₃), —NHSO₂CH₂CH₂NH₂, —NHSO₂CH₂CH₂CH₂NH₂, —NHSO₂CH₂CH₂NHCH₃, —NHSO₂CH₂CH₂N(CH₃)₂, —NHSO₂CH₂CH₂NH₂(CH₃)(CH₂CH₃), —NHSO₂CH₂CH₂N(CH₂CH₃)(CH₂CH₃), —NHCH₂CH₂NH₂, —NHCH₂NH₂, —NHCH₂CH₂CH₂NH₂, —NHCH₂CH₂CH₂CH₂NH₂, —NHCH₂CH₂NHCH₃, —NHCH₂CH₂N(CH₃)₂, —N(CH₂CH₂NH₂)₂, —N(CH₂CH₂CH₂NH₂)₂, —N(CH₂NH₂)₂, —N(CH₂NHCH₃)₂, —N(CH₂CH₂NHCH₃)₂, —N(CH₂CH₂CH₂NHCH₃)₂, —NHCONHCH₂CH₂SO₂NH₂, —NHCONHCH₂CH₂CH₂SO₂NH₂, —NHCON(CH₃)(CH₂CH₂CH₂SO₂NH₂), —NHCON(CH₂CH₃)(CH₂CH₂CH₂SO₂NH₂), —NHCON(CH₃)(CH₂CH₂SO₂NH₂), —NHCONHCH₂CH₂SO₂NHCH₃, —NHCONHCH₂CH₂SO₂NHCH₂CH₃, —NHCONHCH₂CH₂SO₂N(CH₃)₂, —NHCH₂CH₂SO₂NH₂, —NHCH₂CH₂CH₂SO₂NH₂, —NHCH₂CH₂CH₂CH₂SO₂NH₂, —NHCH₂CH₂SO₂NHCH₃, —NHCH₂CH₂SO₂N(CH₃)₂.

According to a preferred embodiment of the invention, in the molecules shown with Formula I, when n=0, R₄, and R₅ are empty.

Another aspect of the invention are molecules shown with Formula II

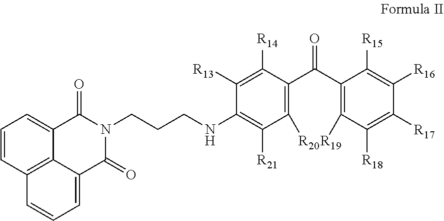

Wherein R₁₃, R₁₄, R₁₅, R₁₆, R₁₇, R₁₈, R₁₉, R₂₀, R₂₁ are independently selected from; —H, —Cl, —F, —Br, —I, R_{X}, —NH₂, —NHR_{X}, —NR_{X}R_{Y}, —OH, —NO, —NO₂, —COOH, —OR_{X}, —COOR_{X}, —CN and

R_{X} and R_{Y} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or hetero-aromatic rings that comprise one or more heteroatoms selected from O, S and N.

According to a preferred embodiment of the invention the molecules according to the invention are illustrated with Formula II;

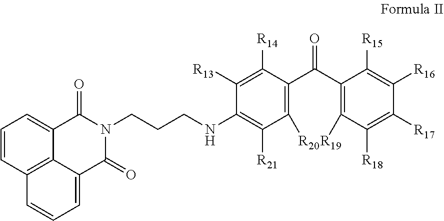

Wherein R₁₃, R₁₄, R₁₅, R₁₆, R₁₇, R₁₈, R₁₉, R₂₀, R₂₁ are independently selected from the group of; —H, —Cl, —F, —Br, —I, —NH₂, —CH₃, C(CH₃)₂. Another aspect of the invention are molecules shown with Formula III

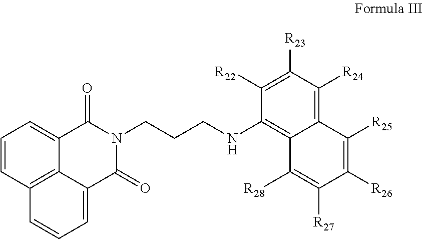

Wherein R₂₂, R₂₃, R₂₄, R₂₅, R₂₆, R₂₇, R₂₈, are independently selected from the group of; —H, —Cl, —F, —Br, —I, R_{X}, —NH₂, —NHR_{X}, —NR_{X}R_{Y}, —OH, —COOH, —OR_{X}, —COOR_{X}, —CN, —SO₃H, —SO₃⁻Na⁺,

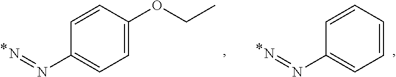

### −NO and

R_{X} and R_{Y} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

Another aspect of the invention are molecules shown with Formula IV

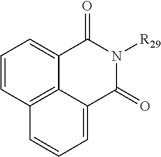

Formula IV is selected from the group formed of R₂₉

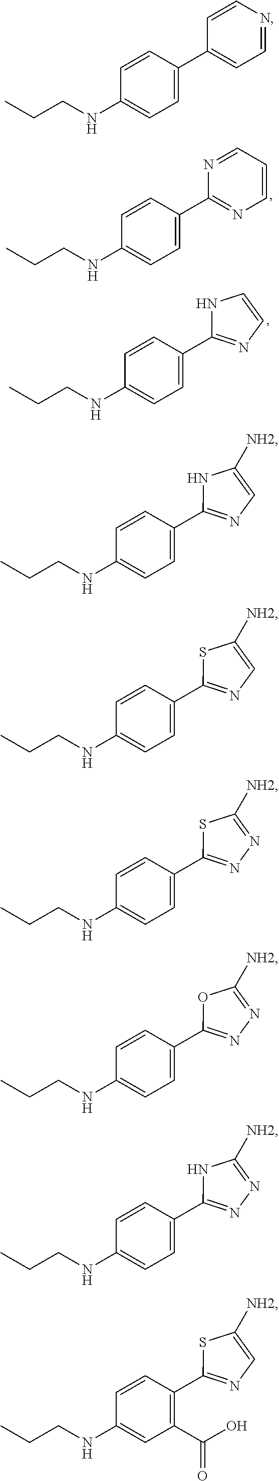

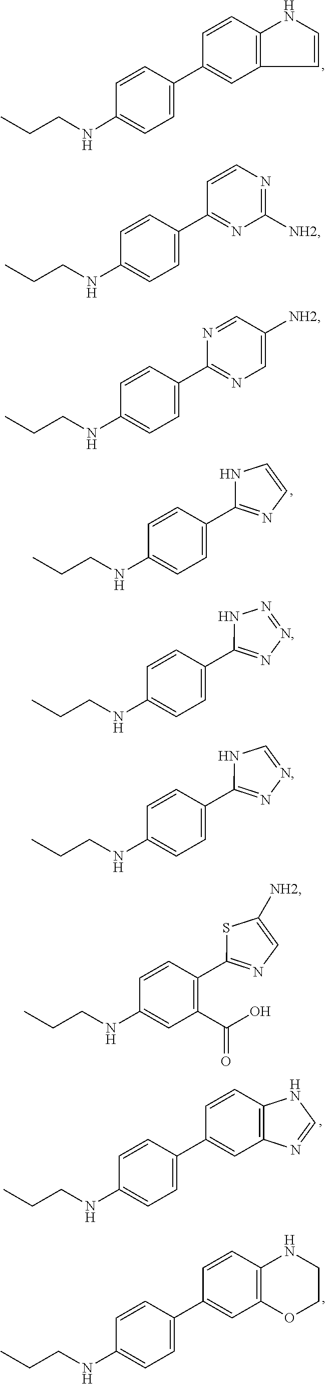

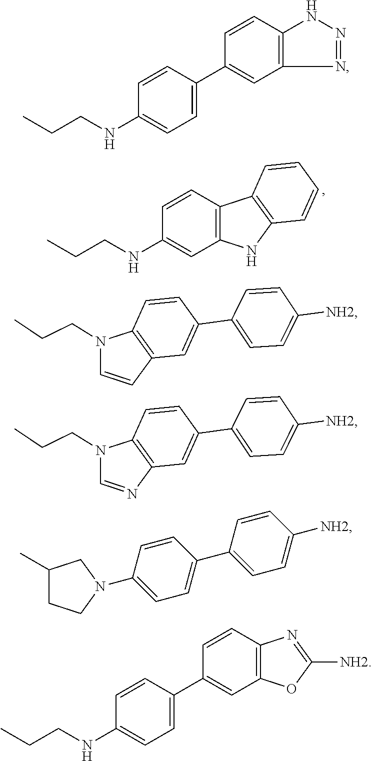

Another aspect of the invention are molecules shown with Formula V

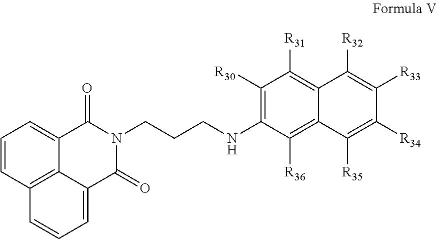

Wherein R₃₀, R₃₁, R₃₂, R₃₃, R₃₄, R₃₅, R₃₆ are independently selected from the group of; —H, —Cl, —F, —Br, —I, R_{X}, —NH₂, —NHR_{X}, —NR_{X}R_{Y}, —OH, —NO, —COOH, —OR_{X}, —COOR_{X}, —CN and

R_{X} and R_{Y} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

According to a preferred embodiment of the invention the molecules according to the invention are illustrated with Formula V;

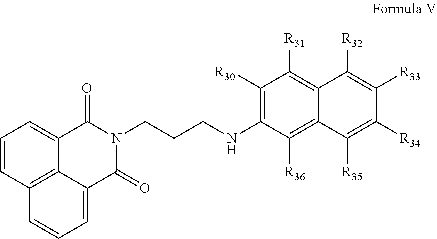

wherein R₃₀, R₃₁, R₃₂, R₃₃, R₃₄, R₃₅, R₃₆ are independently selected from the group of; —H, —NH₂, —OCH₃, —NO, —Cl, F, I, Br.

Another aspect of the invention are molecules shown with Formula VI;

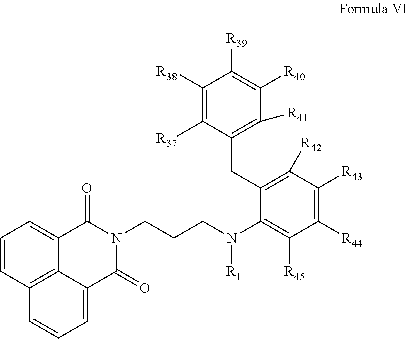

wherein R₁, R₃₇, R₃₈, R₃₉, R₄₀, R₄₁, R₄₂, R₄₃, R₄₄, R₄₅ are independently selected from the group of;
—H, —NH₂, —NHR_{X}, —NR_{X}R_{Y}, —NHNR_{X}, —N(R_{X})(NHR_{Y}), —N(R_{X})(NR_{Y}R_{Z}), R_{X}, R_{Y}, R_{Z}, Cl, F, Br, I, —OH, —NO, —NO₂, —COOH, —OR_{X}, —SO₂R_{X}, —SO₂NHR_{X}, —SO₂NR_{X}R_{Y}, —CN, —COOR_{X}, —CONHR_{X}, —CONR_{X}R_{Y}, —NHCOCH₂R_{X}, —NHCOCHR_{X}R_{Y}, —NHCNHR_{X}, —NHCOR_{X}, —NHCONHR_{X}, —NHSO₂R_{X} and;
R_{X}, R_{Y}, and R_{Z} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

According to a preferred embodiment of the invention the molecules according to the invention are illustrated with Formula VI;

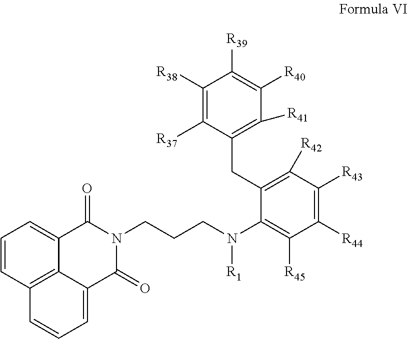

wherein R₁, R₃₇, R₃₈, R₃₉, R₄₀, R₄₁, R₄₂, R₄₃, R₄₄, R₄₅ are independently selected from the group of;
—H, —NH₂, —CH₃, —CH₂CH₃, —CH₂CH₂CH₃, —C(CH₃)₃, Cl, F, Br, I, —OH, —OCH₃, —OCH₂CH₃, —NHCH₃, —NHCH₂CH₃, —N(CH₃)₂, —NO₂, —COOH, —COOCH₃, —COONH₂, —COONHCH₃, —COON(CH₃)₂.

Another aspect of the invention are molecules shown with Formula VII;

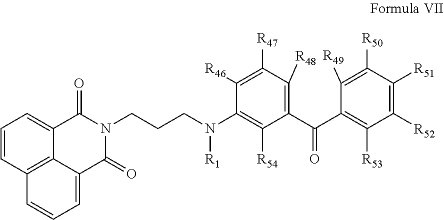

where R₁, R₄₆, R₄₇, R₄₈, R₄₉, R₅₀, R₅₁, R₅₂, R₅₃, R₅₄ is selected from the group of; —H, —NH₂, —NHR_{X}, —NR_{X}R_{Y}, —NHNR_{X}, —N(R_{X})(NHR_{Y}), —N(R_{X})(NR_{Y}R_{Z}), R_{X}, R_{Y}, R_{Z}, Cl, F, Br, I, —OH, —NO, —NO₂, —COOH, —OR_{X}, —SO₂R_{X}, —SO₂NHR_{X}, —SO₂NR_{X}R_{Y}, —CN, —COOR_{X}, —CONHR_{X}, —CONR_{X}R_{Y}, —NHCOCH₂R_{X}, —NHCOCHR_{X}R_{Y}, —NHCNHR_{X}, —NHCOR_{X}, —NHCONHR_{X}, —NHSO₂R_{X} and;
R_{X}, R_{Y}, and R_{Z} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

According to a preferred embodiment of the invention the molecules according to the invention are illustrated with Formula VII;

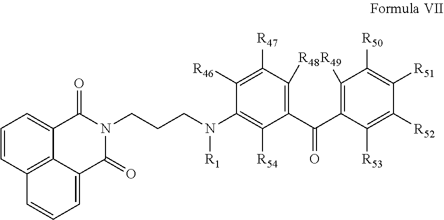

wherein R₁, R₄₆, R₄₇, R₄₈, R₄₉, R₅₀, R₅₁, R₅₂, R₅₃, R₅₄ is selected from the group of; —H, —NH₂, —CH₃, —CH₂CH₃, —CH₂CH₂CH₃, —C(CH₃)₃, Cl, F, Br, I, —OH, —OCH₃, —OCH₂CH₃, —NHCH₃, —NHCH₂CH₃, —N(CH₃)₂, —NO₂, —COOH, —COOCH₃, —COONH₂, —COONHCH₃, —COON(CH₃)₂.

According to a preferred embodiment of the invention R₁ in the molecules suitable to Formula VII is —H.

Another aspect of the invention are molecules shown with Formula VIII;

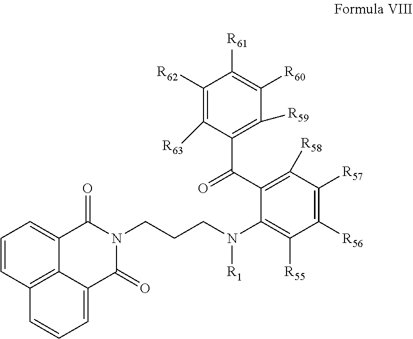

wherein R₁, R₅₅, R₅₆, R₅₇, R₅₈, R₅₉, R₆₀, R₆₁, R₆₂, R₆₃ are independently selected from the group of,
—H, —NH₂, —NHR_{X}, —NR_{X}R_{Y}, —NHNR_{X}, —N(R_{X})(NHR_{Y}), —N(R_{X})(NR_{Y}R_{Z}), R_{X}, R_{Y}, R_{Z}, Cl, F, Br, I, —OH, —NO, —NO₂, —COOH, —OR_{X}, —SO₂R_{X}, —SO₂NHR_{X}, —SO₂NR_{X}R_{Y}, —CN, —COOR_{X}, —CONHR_{X}, —CONR_{X}R_{Y}, —NHCOCH₂R_{X}, —NHCOCHR_{X}R_{Y}, —NHCNHR_{X}, —NHCOR_{X}, —NHCONHR_{X}, —NHSO₂R_{X} and;
R_{X}, R_{Y}, and R_{Z} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

According to a preferred embodiment of the invention the molecules according to the invention are illustrated with Formula VIII;

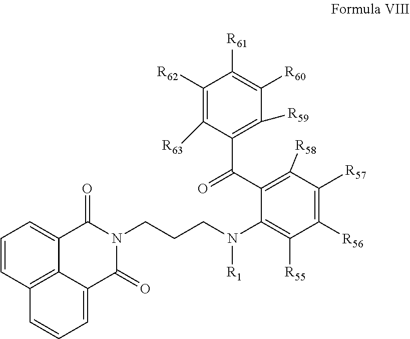

wherein R₁, R₅₅, R₅₆, R₅₇, R₅₈, R₅₉, R₆₀, R₆₁, R₆₂, R₆₃ are independently selected from the group of;
—H, —NH₂, —CH₃, —CH₂CH₃, —CH₂CH₂CH₃, —C(CH₃)₃, Cl, F, Br, I, —OH, —OCH₃, —OCH₂CH₃, —NHCH₃, —NHCH₂CH₃, —N(CH₃)₂, —NO₂, —COOH, —COOCH₃, —COONH₂, —COONHCH₃, —COON(CH₃)₂.

According to a preferred embodiment of the invention R₁ in the molecules suitable to Formula VIII is —H.

The present invention is related to bisnaphthalimidopropyl derived molecules, wherein molecules suitable to the invention are shown with;

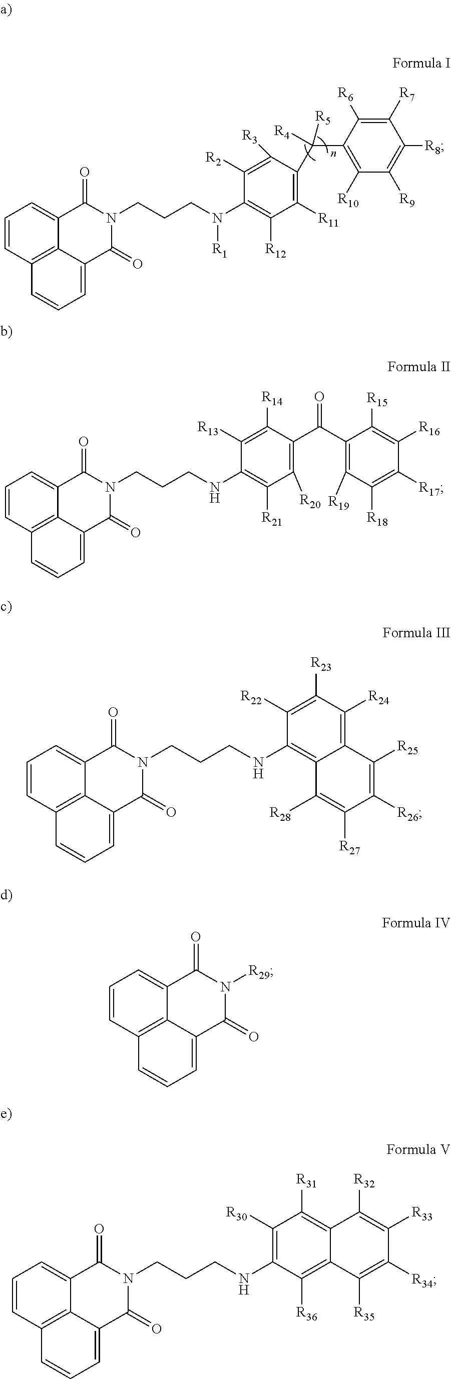

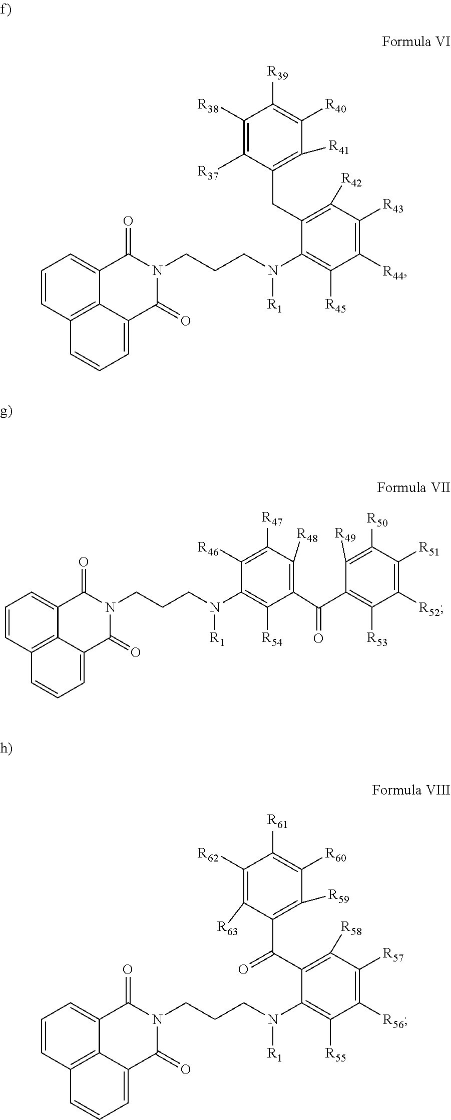

wherein
-n is a natural number between 0 to 12,
—R₁, R₂, R₃, R₄, R₅, R₆, R₇, R₈, R₉, R₁₀, R₁₁, R₁₂, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, are independently selected from the group of;
—H, —NH₂, —NHR_{X}, —NR_{X}R_{Y}, —NHNR_{X}, —N(R_{X})(NHR_{Y}), —N(R_{X})(NR_{Y}R_{Z}), R_{X}, R_{Y}, R_{Z}, Cl, F, Br, I, —OH, —NO, —NO₂, —COOH, —OR_{X}, —SO₂R_{X}, —SO₂NHR_{X}, —SO₂NR_{X}R_{Y}, —CN, —COOR_{X}, —CONHR_{X}, —CONR_{X}R_{Y}, —NHCOCH₂R_{X}, —NHCOCHR_{X}R_{Y}, —NHCNHR_{X}, —NHCOR_{X}, —NHCONHR_{X}, —NHSO₂R_{X};
—R₁₃, R₁₄, R₁₅, R₁₆, R₁₇, R₁₈, R₁₉, R₂₀, R₂₁ is independently selected from;
—H, —Cl, —F, —Br, —I, R_{X}, —NH₂, —NHR_{X}, —NR_{X}R_{Y}, —OH, —NO, —NO₂, —COOH, —OR_{X}, —COOR_{X}, —CN;
—R₂₂, R₂₃, R₂₄, R₂₅, R₂₆, R₂₇, R₂₈ are independently selected from;
—H, —Cl, —F, —Br, —I, R_{X}, —NH₂, —NHR_{X}, —NR_{X}R_{Y}, —OH, —COOH, —OR_{X}, —COOR_{X}, —CN, —SO₃H, —SO₃⁻Na⁺,

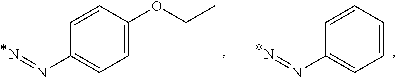

### —NO;

—R₂₉ is selected from the group of;

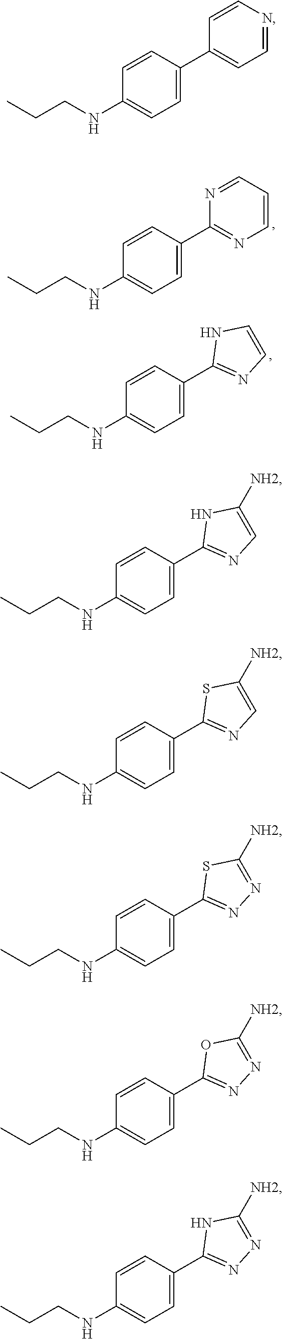

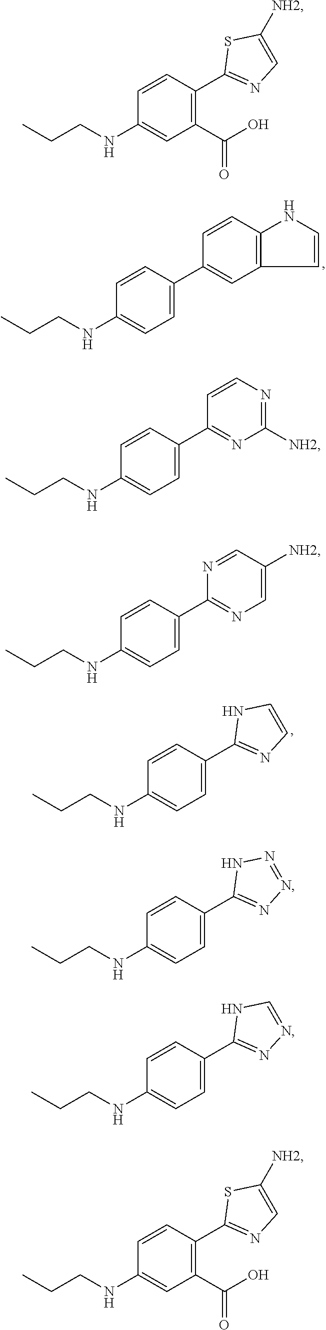

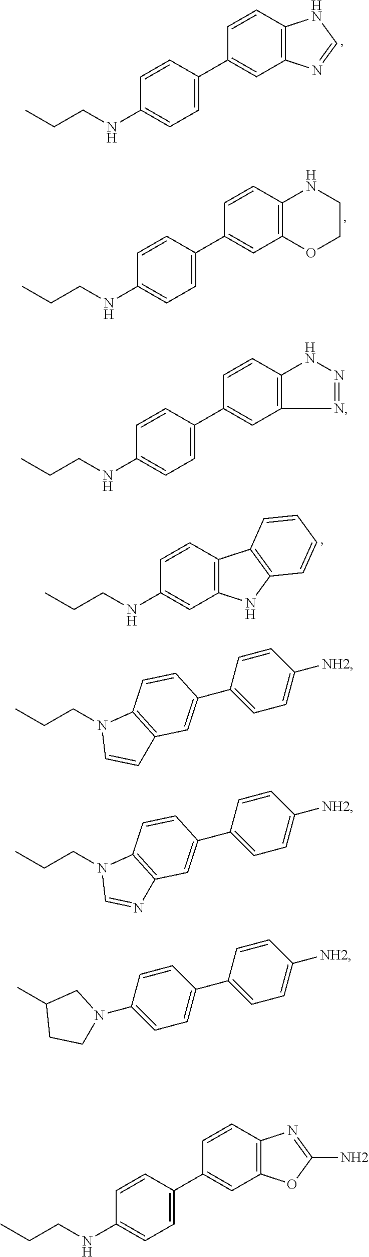

—R₃₀, R₃₁, R₃₂, R₃₃, R₃₄, R₃₅, R₃₆ are independently selected from the group of;
—H, —Cl, —F, —Br, —I, R_{X}, —NH₂, —NHR_{X}, —NR_{X}R_{Y}, —OH, —NO, —COOH, —OR_{X}, —COOR_{X}, —CN;
R_{X}, R_{Y}, and R_{Z} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

According to a preferred embodiment of the invention the molecule suitable to Formula I is illustrated with Formula 1;

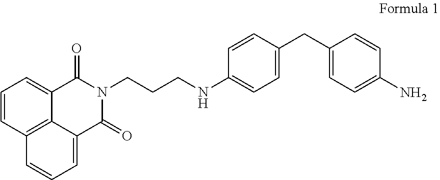 [SMILES: Nc1ccc(Cc2ccc(NCCCN3C(=O)c4cccc5cccc(c45)C3=O)cc2)cc1]

According to a preferred embodiment of the invention the molecule suitable to Formula I is illustrated with Formula 2;

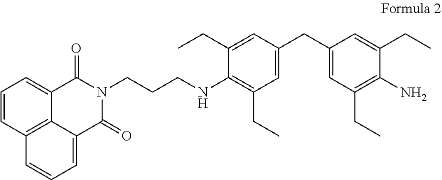 [SMILES: CCc1cc(Cc2cc(CC)c(NCCCN3C(=O)c4cccc5cccc(c45)C3=O)c(CC)c2)cc(CC)c1N]

According to a preferred embodiment of the invention the molecule suitable to Formula I is illustrated with Formula 3;

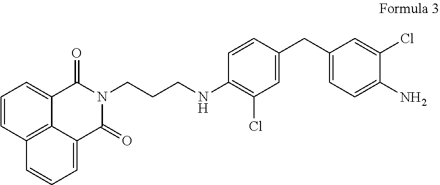 [SMILES: Nc1ccc(Cc2ccc(NCCCN3C(=O)c4cccc5cccc(c45)C3=O)c(Cl)c2)cc1Cl]

According to a preferred embodiment of the invention the molecule suitable to Formula I is illustrated with Formula 4;

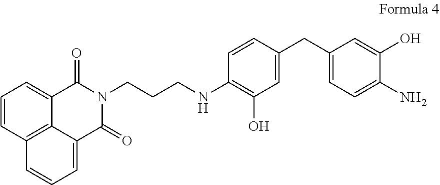 [SMILES: Nc1ccc(Cc2ccc(NCCCN3C(=O)c4cccc5cccc(c45)C3=O)c(O)c2)cc1O]

According to a preferred embodiment of the invention the molecule suitable to Formula I is illustrated with Formula 5;

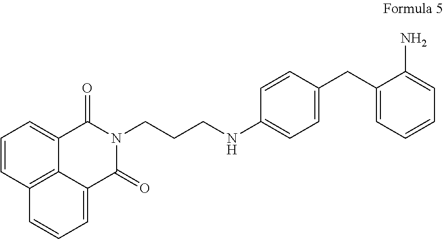 [SMILES: Nc1ccccc1Cc1ccc(NCCCN2C(=O)c3cccc4cccc(c34)C2=O)cc1]

According to a preferred embodiment of the invention the molecule suitable to Formula I is illustrated with Formula 6;

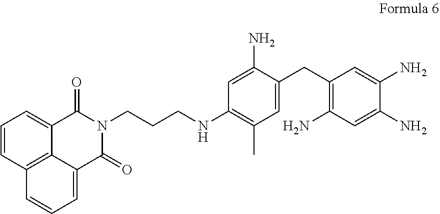 [SMILES: Cc1cc(Cc2cc(N)c(N)cc2N)c(N)cc1NCCCN1C(=O)c2cccc3cccc(c23)C1=O]

According to a preferred embodiment of the invention the molecule suitable to Formula I is illustrated with Formula 7;

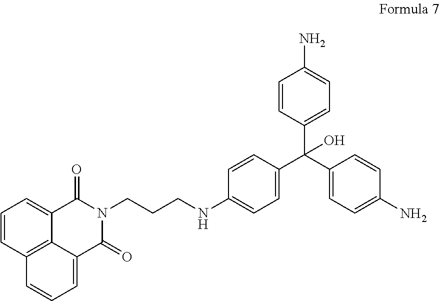 [SMILES: Nc1ccc(C(O)(c2ccc(N)cc2)c2ccc(NCCCN3C(=O)c4cccc5cccc(c45)C3=O)cc2)cc1]

According to a preferred embodiment of the invention the molecule suitable to Formula I is illustrated with Formula 8;

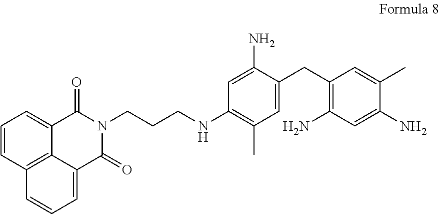 [SMILES: Cc1cc(Cc2cc(C)c(NCCCN3C(=O)c4cccc5cccc(c45)C3=O)cc2N)c(N)cc1N]

According to a preferred embodiment of the invention the molecule suitable to Formula I is illustrated with Formula 9;

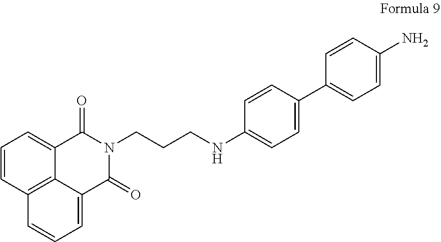 [SMILES: Nc1ccc(-c2ccc(NCCCN3C(=O)c4cccc5cccc(c45)C3=O)cc2)cc1]

According to a preferred embodiment of the invention the molecule suitable to Formula I is illustrated with Formula 10;

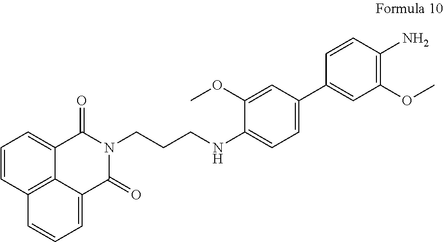 [SMILES: COc1cc(-c2ccc(NCCCN3C(=O)c4cccc5cccc(c45)C3=O)c(OC)c2)ccc1N]

According to a preferred embodiment of the invention the molecule suitable to Formula I is illustrated with Formula 11;

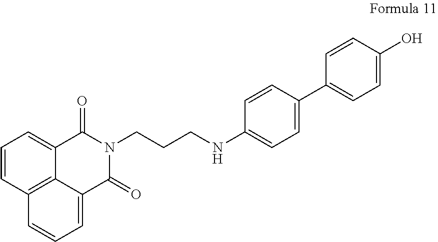 [SMILES: O=C1c2cccc3cccc(c23)C(=O)N1CCCNc1ccc(-c2ccc(O)cc2)cc1]

According to a preferred embodiment of the invention the molecule suitable to Formula I is illustrated with Formula 12;

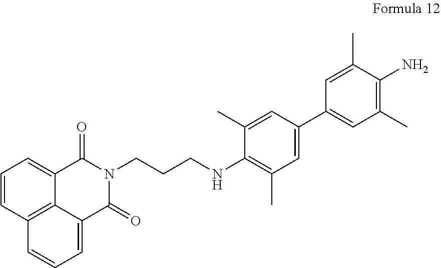 [SMILES: Cc1cc(-c2cc(C)c(NCCCN3C(=O)c4cccc5cccc(c45)C3=O)c(C)c2)cc(C)c1N]

According to a preferred embodiment of the invention the molecule suitable to Formula I is illustrated with Formula 13;

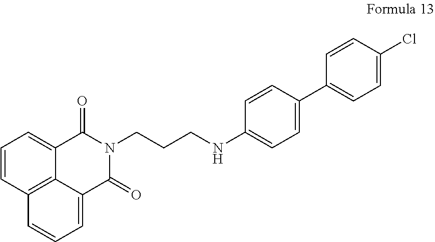 [SMILES: O=C1c2cccc3cccc(c23)C(=O)N1CCCNc1ccc(-c2ccc(Cl)cc2)cc1]

According to a preferred embodiment of the invention the molecule suitable to Formula I is illustrated with Formula 14;

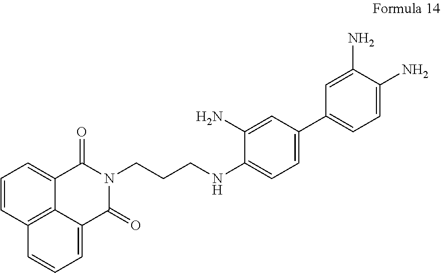 [SMILES: Nc1ccc(-c2ccc(NCCCN3C(=O)c4cccc5cccc(c45)C3=O)c(N)c2)cc1N]

According to a preferred embodiment of the invention the molecule suitable to Formula I is illustrated with Formula 15;

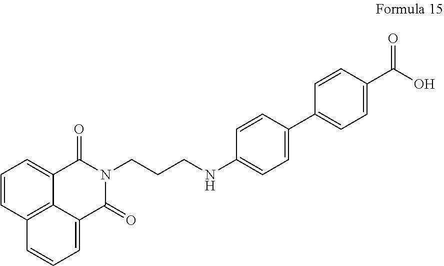 [SMILES: O=C(O)c1ccc(-c2ccc(NCCCN3C(=O)c4cccc5cccc(c45)C3=O)cc2)cc1]

According to a preferred embodiment of the invention the molecule suitable to Formula I is illustrated with Formula 16;

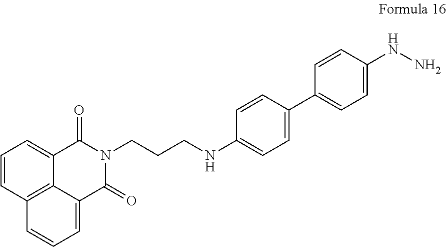 [SMILES: NNc1ccc(-c2ccc(NCCCN3C(=O)c4cccc5cccc(c45)C3=O)cc2)cc1]

According to a preferred embodiment of the invention the molecule suitable to Formula I is illustrated with Formula 17;

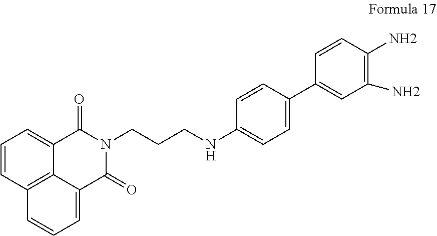 [SMILES: Nc1ccc(-c2ccc(NCCCN3C(=O)c4cccc5cccc(c45)C3=O)cc2)cc1N]

According to a preferred embodiment of the invention the molecule suitable to Formula I is illustrated with Formula 18;

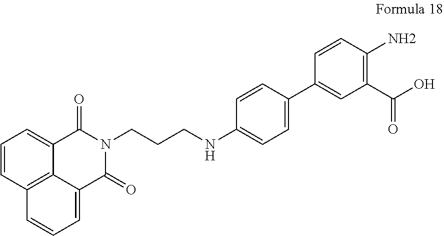 [SMILES: Nc1ccc(-c2ccc(NCCCN3C(=O)c4cccc5cccc(c45)C3=O)cc2)cc1C(=O)O]

According to a preferred embodiment of the invention the molecule suitable to Formula I is illustrated with Formula 19;

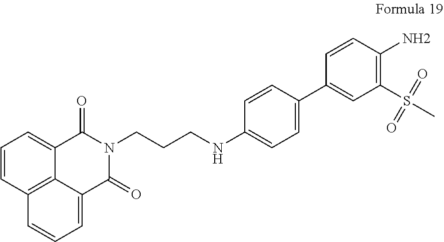 [SMILES: CS(=O)(=O)c1cc(-c2ccc(NCCCN3C(=O)c4cccc5cccc(c45)C3=O)cc2)ccc1N]

According to a preferred embodiment of the invention the molecule suitable to Formula I is illustrated with Formula 20;

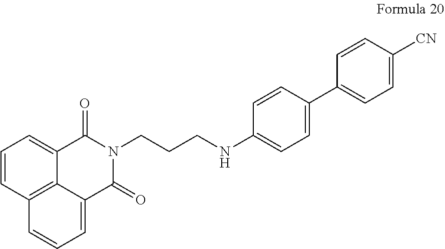 [SMILES: N#Cc1ccc(-c2ccc(NCCCN3C(=O)c4cccc5cccc(c45)C3=O)cc2)cc1]

According to a preferred embodiment of the invention the molecule suitable to Formula I is illustrated with Formula 21;

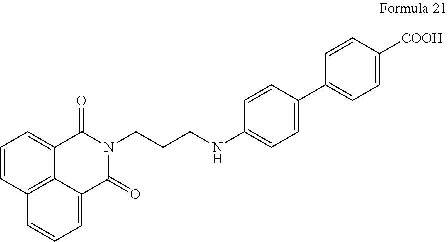 [SMILES: O=C(O)c1ccc(-c2ccc(NCCCN3C(=O)c4cccc5cccc(c45)C3=O)cc2)cc1]

According to a preferred embodiment of the invention the molecule suitable to Formula I is illustrated with Formula 22;

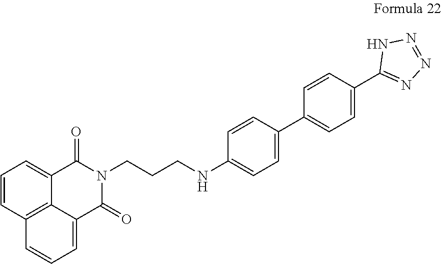 [SMILES: O=C1c2cccc3cccc(c23)C(=O)N1CCCNc1ccc(-c2ccc(-c3nnn[nH]3)cc2)cc1]

According to a preferred embodiment of the invention the molecule suitable to Formula I is illustrated with Formula 23;

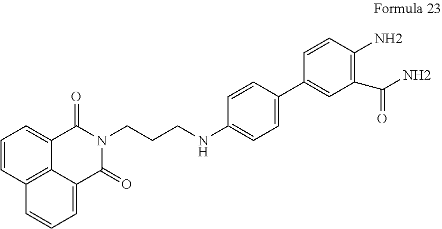 [SMILES: NC(=O)c1cc(-c2ccc(NCCCN3C(=O)c4cccc5cccc(c45)C3=O)cc2)ccc1N]

According to a preferred embodiment of the invention the molecule suitable to Formula I is illustrated with Formula 24;

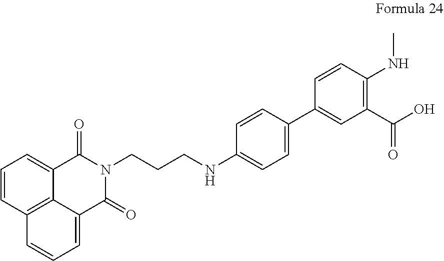 [SMILES: CNc1ccc(-c2ccc(NCCCN3C(=O)c4cccc5cccc(c45)C3=O)cc2)cc1C(=O)O]

According to a preferred embodiment of the invention the molecule suitable to Formula I is illustrated with Formula 25;

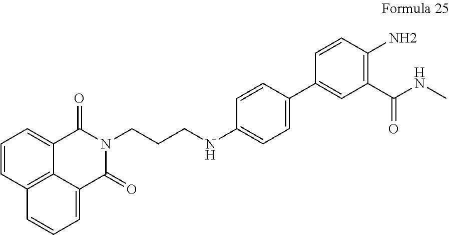 [SMILES: CNC(=O)c1cc(-c2ccc(NCCCN3C(=O)c4cccc5cccc(c45)C3=O)cc2)ccc1N]

According to a preferred embodiment of the invention the molecule suitable to Formula I is illustrated with Formula 26;

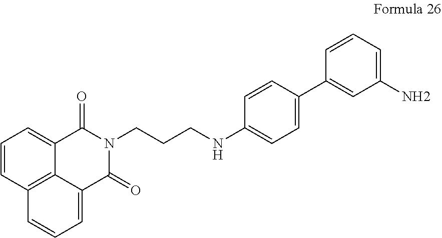 [SMILES: Nc1cccc(-c2ccc(NCCCN3C(=O)c4cccc5cccc(c45)C3=O)cc2)c1]

According to a preferred embodiment of the invention the molecule suitable to Formula I is illustrated with Formula 27;

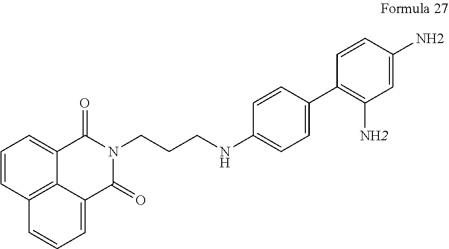 [SMILES: Nc1ccc(-c2ccc(NCCCN3C(=O)c4cccc5cccc(c45)C3=O)cc2)c(N)c1]

According to a preferred embodiment of the invention the molecule suitable to Formula I is illustrated with Formula 28;

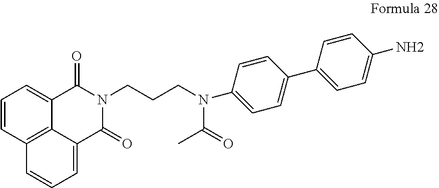 [SMILES: CC(=O)N(CCCN1C(=O)c2cccc3cccc(c23)C1=O)c1ccc(-c2ccc(N)cc2)cc1]

According to a preferred embodiment of the invention the molecule suitable to Formula I is illustrated with Formula 29;

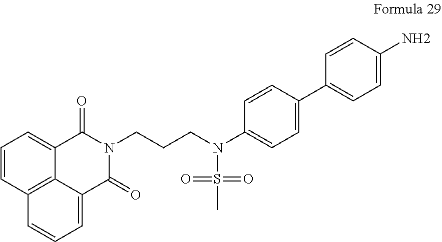 [SMILES: CS(=O)(=O)N(CCCN1C(=O)c2cccc3cccc(c23)C1=O)c1ccc(-c2ccc(N)cc2)cc1]

According to a preferred embodiment of the invention the molecule suitable to Formula I is illustrated with Formula 30;

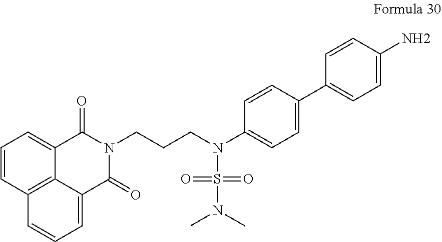 [SMILES: CN(C)S(=O)(=O)N(CCCN1C(=O)c2cccc3cccc(c23)C1=O)c1ccc(-c2ccc(N)cc2)cc1]

According to a preferred embodiment of the invention the molecule suitable to Formula I is illustrated with Formula 31;

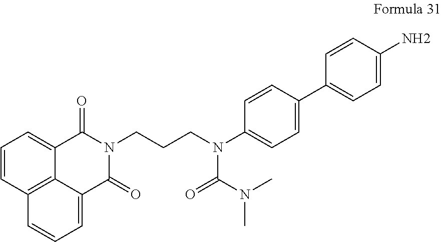 [SMILES: CN(C)C(=O)N(CCCN1C(=O)c2cccc3cccc(c23)C1=O)c1ccc(-c2ccc(N)cc2)cc1]

According to a preferred embodiment of the invention the molecule suitable to Formula I is illustrated with Formula 32;

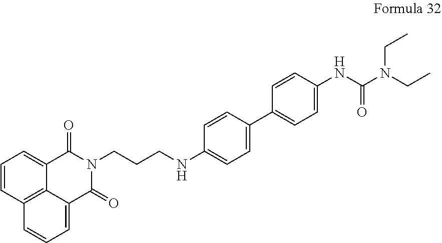 [SMILES: CCN(CC)C(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 33;

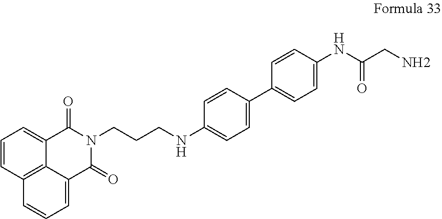 [SMILES: NCC(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 34;

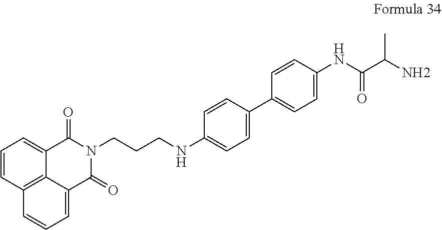 [SMILES: CC(N)C(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 35;

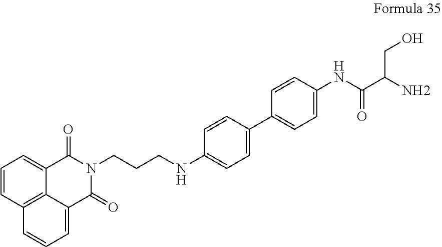 [SMILES: NC(CO)C(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 36;

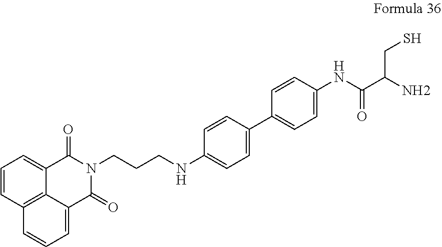 [SMILES: NC(CS)C(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 37;

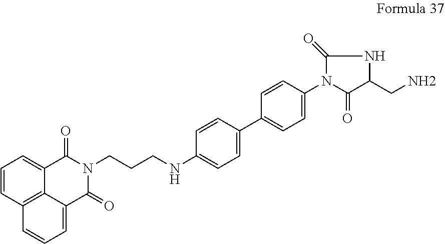 [SMILES: NCC1NC(=O)N(c2ccc(-c3ccc(NCCCN4C(=O)c5cccc6cccc(c56)C4=O)cc3)cc2)C1=O]

According to a preferred embodiment of the invention the molecule suitable to Formula I is illustrated with Formula 38;

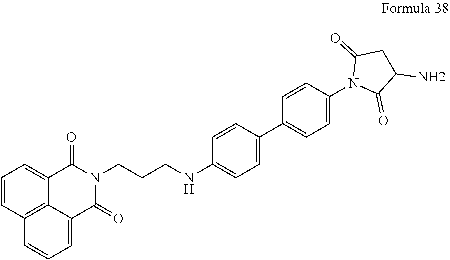 [SMILES: NC1CC(=O)N(c2ccc(-c3ccc(NCCCN4C(=O)c5cccc6cccc(c56)C4=O)cc3)cc2)C1=O]

According to a preferred embodiment of the invention the molecule suitable to Formula I is illustrated with Formula 39;

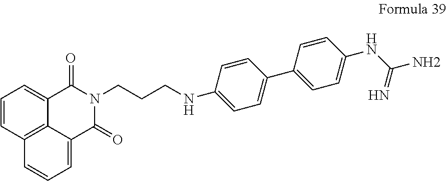 [SMILES: N=C(N)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 40;

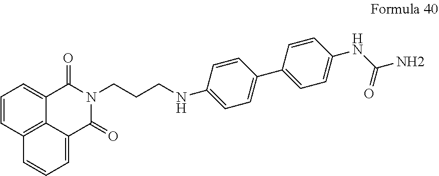 [SMILES: NC(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 41;

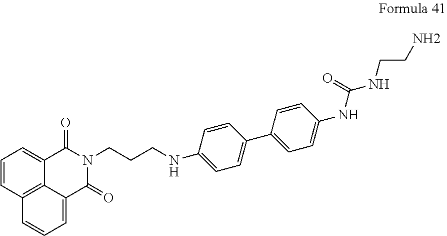 [SMILES: NCCNC(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 42;

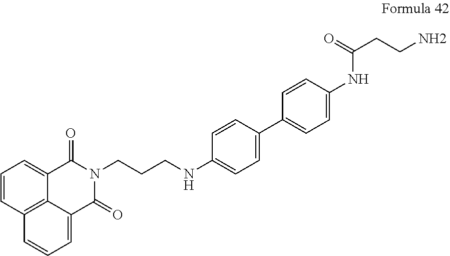 [SMILES: NCCC(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 43;

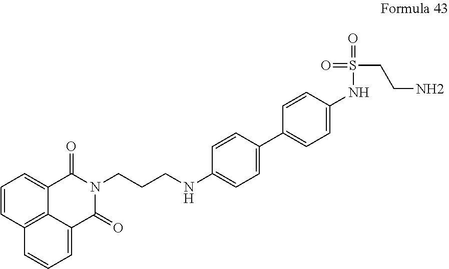 [SMILES: NCCS(=O)(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 44;

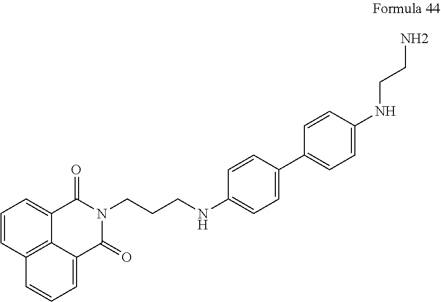 [SMILES: NCCNc1ccc(-c2ccc(NCCCN3C(=O)c4cccc5cccc(c45)C3=O)cc2)cc1]

According to a preferred embodiment of the invention the molecule suitable to Formula I is illustrated with Formula 45;

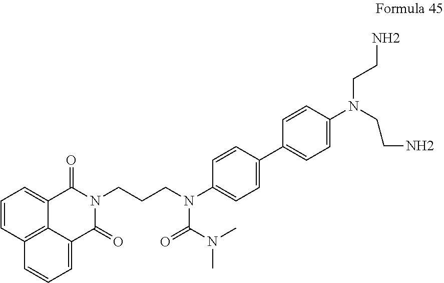 [SMILES: CN(C)C(=O)N(CCCN1C(=O)c2cccc3cccc(c23)C1=O)c1ccc(-c2ccc(N(CCN)CCN)cc2)cc1]

According to a preferred embodiment of the invention the molecule suitable to Formula I is illustrated with Formula 46;

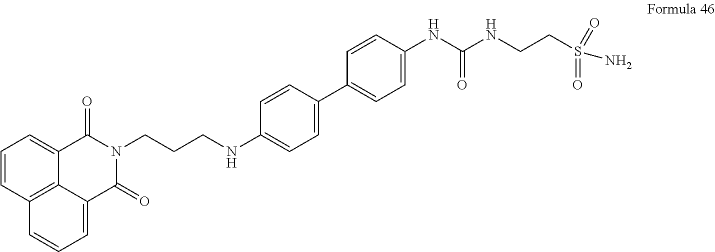 [SMILES: NS(=O)(=O)CCNC(=O)Nc1ccc(-c2ccc(NCCCN3C(=O)c4cccc5cccc(c45)C3=O)cc2)cc1]

According to a preferred embodiment of the invention the molecule suitable to Formula I is illustrated with Formula 47;

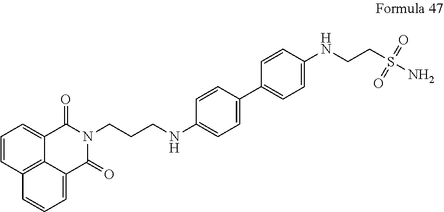 [SMILES: NS(=O)(=O)CCNc1ccc(-c2ccc(NCCCN3C(=O)c4cccc5cccc(c45)C3=O)cc2)cc1]

According to a preferred embodiment of the invention the molecule suitable to Formula II is illustrated with Formula 48;

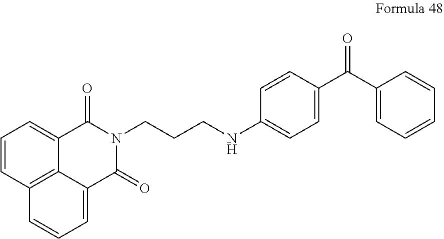 [SMILES: O=C(c1ccccc1)c1ccc(NCCCN2C(=O)c3cccc4cccc(c34)C2=O)cc1]

According to a preferred embodiment of the invention the molecule suitable to Formula II is illustrated with Formula 49;

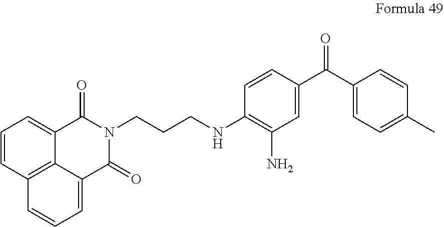 [SMILES: Cc1ccc(C(=O)c2ccc(NCCCN3C(=O)c4cccc5cccc(c45)C3=O)c(N)c2)cc1]

According to a preferred embodiment of the invention the molecule suitable to Formula II is illustrated with Formula 50;

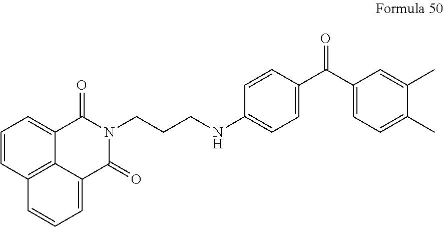 [SMILES: Cc1ccc(C(=O)c2ccc(NCCCN3C(=O)c4cccc5cccc(c45)C3=O)cc2)cc1C]

According to a preferred embodiment of the invention the molecule suitable to Formula II is illustrated with Formula 51;

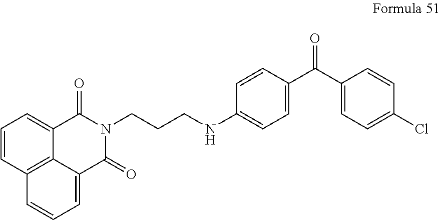 [SMILES: O=C(c1ccc(Cl)cc1)c1ccc(NCCCN2C(=O)c3cccc4cccc(c34)C2=O)cc1]

According to a preferred embodiment of the invention the molecule suitable to Formula II is illustrated with Formula 52;

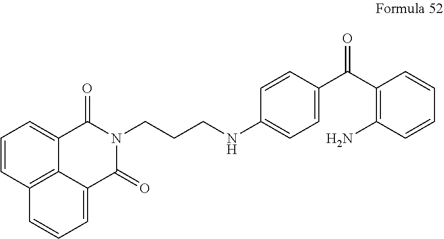 [SMILES: Nc1ccccc1C(=O)c1ccc(NCCCN2C(=O)c3cccc4cccc(c34)C2=O)cc1]

According to a preferred embodiment of the invention the molecule suitable to Formula II is illustrated with Formula 53;

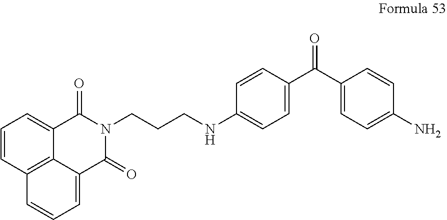 [SMILES: Nc1ccc(C(=O)c2ccc(NCCCN3C(=O)c4cccc5cccc(c45)C3=O)cc2)cc1]

According to a preferred embodiment of the invention the molecule suitable to Formula II is illustrated with Formula 54;

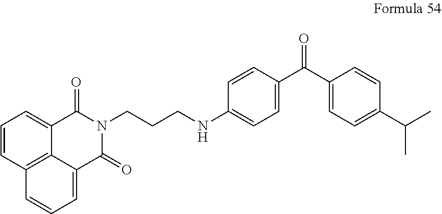 [SMILES: CC(C)c1ccc(C(=O)c2ccc(NCCCN3C(=O)c4cccc5cccc(c45)C3=O)cc2)cc1]

According to a preferred embodiment of the invention the molecule suitable to Formula III is illustrated with Formula 55;

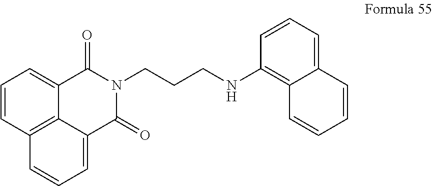 [SMILES: O=C1c2cccc3cccc(c23)C(=O)N1CCCNc1cccc2ccccc12]

According to a preferred embodiment of the invention the molecule suitable to Formula III is illustrated with Formula 56;

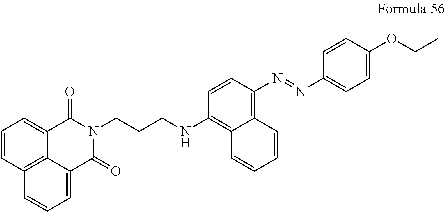 [SMILES: CCOc1ccc(/N=N/c2ccc(NCCCN3C(=O)c4cccc5cccc(c45)C3=O)c3ccccc23)cc1]

According to a preferred embodiment of the invention the molecule suitable to Formula III is illustrated with Formula 57;

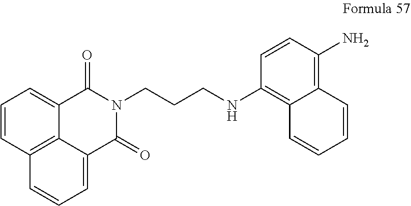 [SMILES: Nc1ccc(NCCCN2C(=O)c3cccc4cccc(c34)C2=O)c2ccccc12]

According to a preferred embodiment of the invention the molecule suitable to Formula III is illustrated with Formula 58;

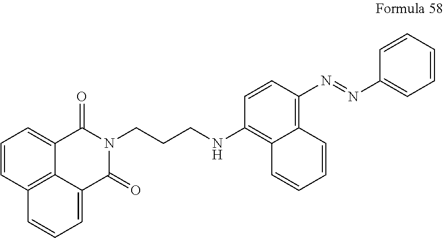 [SMILES: O=C1c2cccc3cccc(c23)C(=O)N1CCCNc1ccc(/N=N/c2ccccc2)c2ccccc12]

According to a preferred embodiment of the invention the molecule suitable to Formula III is illustrated with Formula 59;

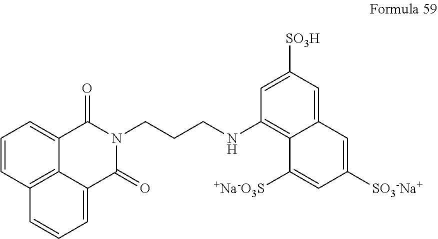

According to a preferred embodiment of the invention the molecule suitable to Formula III is illustrated with Formula 60;

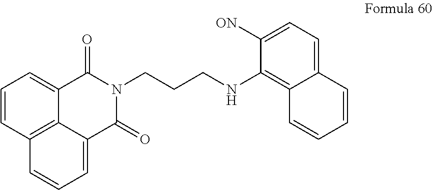

According to a preferred embodiment of the invention the molecule suitable to Formula III is illustrated with Formula 61;

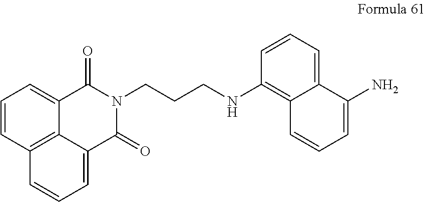 [SMILES: Nc1cccc2c(NCCCN3C(=O)c4cccc5cccc(c45)C3=O)cccc12]

According to a preferred embodiment of the invention the molecule suitable to Formula III is illustrated with Formula 62;

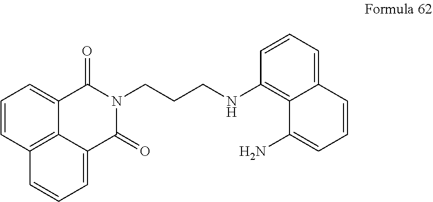 [SMILES: Nc1cccc2cccc(NCCCN3C(=O)c4cccc5cccc(c45)C3=O)c12]

According to a preferred embodiment of the invention the molecule suitable to Formula IV is illustrated with Formula 63;

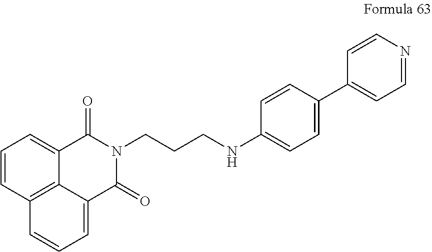 [SMILES: O=C1c2cccc3cccc(c23)C(=O)N1CCCNc1ccc(-c2ccncc2)cc1]

According to a preferred embodiment of the invention the molecule suitable to Formula IV is illustrated with Formula 64;

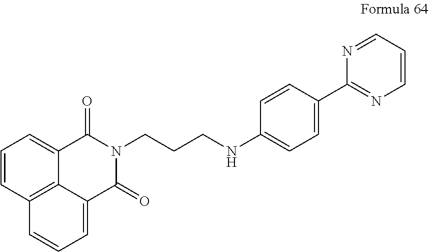 [SMILES: O=C1c2cccc3cccc(c23)C(=O)N1CCCNc1ccc(-c2ncccn2)cc1]

According to a preferred embodiment of the invention the molecule suitable to Formula IV is illustrated with Formula 65;

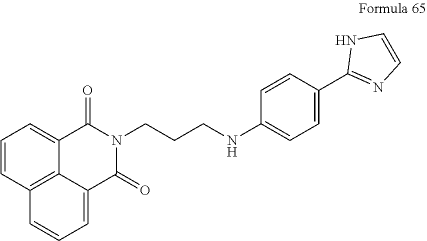 [SMILES: O=C1c2cccc3cccc(c23)C(=O)N1CCCNc1ccc(-c2ncc[nH]2)cc1]

According to a preferred embodiment of the invention the molecule suitable to Formula IV is illustrated with Formula 66;

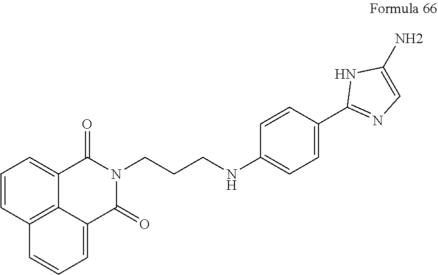 [SMILES: Nc1cnc(-c2ccc(NCCCN3C(=O)c4cccc5cccc(c45)C3=O)cc2)[nH]1]

According to a preferred embodiment of the invention the molecule suitable to Formula IV is illustrated with Formula 67;

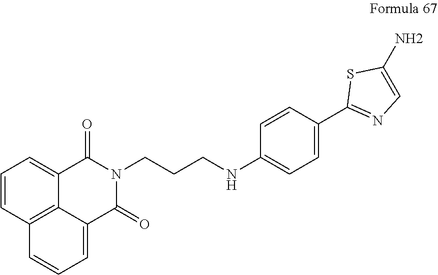 [SMILES: Nc1cnc(-c2ccc(NCCCN3C(=O)c4cccc5cccc(c45)C3=O)cc2)s1]

According to a preferred embodiment of the invention the molecule suitable to Formula IV is illustrated with Formula 68;

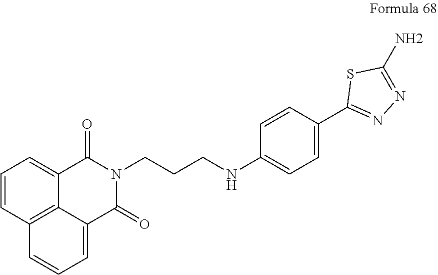 [SMILES: Nc1nnc(-c2ccc(NCCCN3C(=O)c4cccc5cccc(c45)C3=O)cc2)s1]

According to a preferred embodiment of the invention the molecule suitable to Formula IV is illustrated with Formula 69;

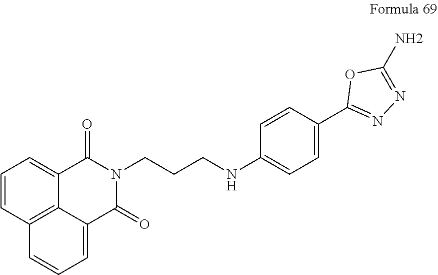 [SMILES: Nc1nnc(-c2ccc(NCCCN3C(=O)c4cccc5cccc(c45)C3=O)cc2)o1]

According to a preferred embodiment of the invention the molecule suitable to Formula IV is illustrated with Formula 70;

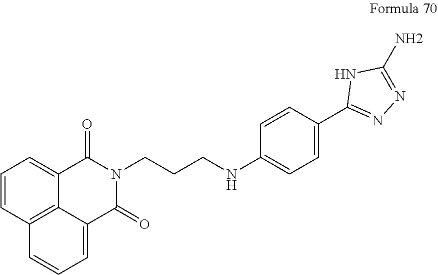 [SMILES: Nc1nnc(-c2ccc(NCCCN3C(=O)c4cccc5cccc(c45)C3=O)cc2)[nH]1]

According to a preferred embodiment of the invention the molecule suitable to Formula IV is illustrated with Formula 71;

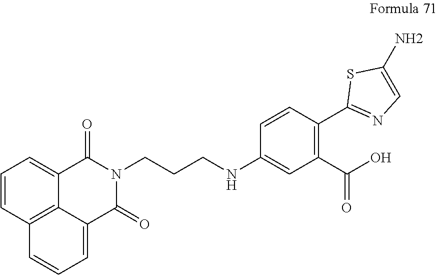 [SMILES: Nc1cnc(-c2ccc(NCCCN3C(=O)c4cccc5cccc(c45)C3=O)cc2C(=O)O)s1]

According to a preferred embodiment of the invention the molecule suitable to Formula IV is illustrated with Formula 72;

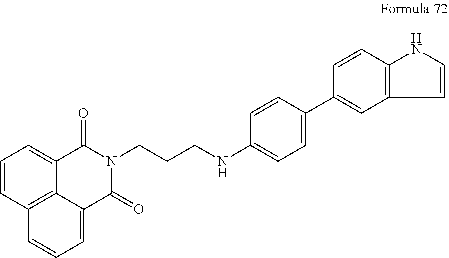 [SMILES: O=C1c2cccc3cccc(c23)C(=O)N1CCCNc1ccc(-c2ccc3[nH]ccc3c2)cc1]

According to a preferred embodiment of the invention the molecule suitable to Formula IV is

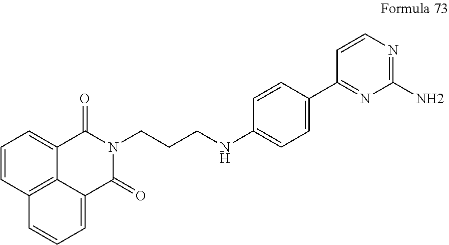 [SMILES: Nc1nccc(-c2ccc(NCCCN3C(=O)c4cccc5cccc(c45)C3=O)cc2)n1]

According to a preferred embodiment of the invention the molecule suitable to Formula IV is illustrated with Formula 74;

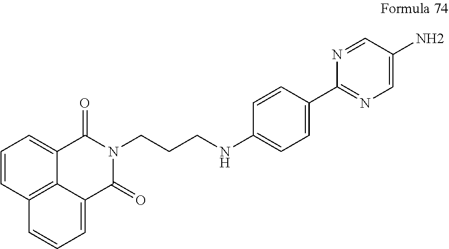 [SMILES: Nc1cnc(-c2ccc(NCCCN3C(=O)c4cccc5cccc(c45)C3=O)cc2)nc1]

According to a preferred embodiment of the invention the molecule suitable to Formula IV is illustrated with Formula 75;

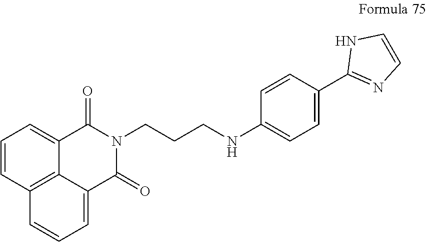 [SMILES: O=C1c2cccc3cccc(c23)C(=O)N1CCCNc1ccc(-c2ncc[nH]2)cc1]

According to a preferred embodiment of the invention the molecule suitable to Formula IV is illustrated with Formula 76;

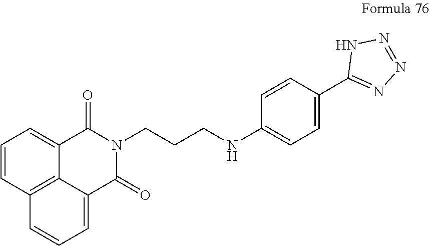 [SMILES: O=C1c2cccc3cccc(c23)C(=O)N1CCCNc1ccc(-c2nnn[nH]2)cc1]

According to a preferred embodiment of the invention the molecule suitable to Formula IV is illustrated with Formula 77;

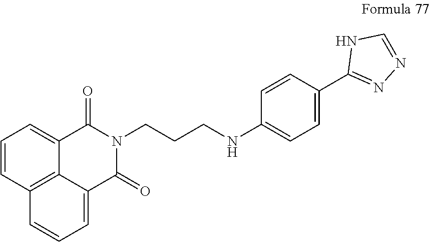 [SMILES: O=C1c2cccc3cccc(c23)C(=O)N1CCCNc1ccc(-c2nnc[nH]2)cc1]

According to a preferred embodiment of the invention the molecule suitable to Formula IV is illustrated with Formula 78;

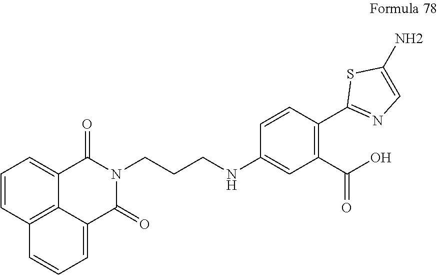 [SMILES: Nc1cnc(-c2ccc(NCCCN3C(=O)c4cccc5cccc(c45)C3=O)cc2C(=O)O)s1]

According to a preferred embodiment of the invention the molecule suitable to Formula IV is illustrated with Formula 79;

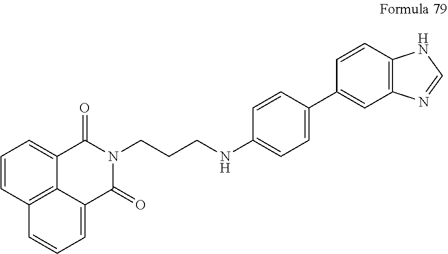 [SMILES: O=C1c2cccc3cccc(c23)C(=O)N1CCCNc1ccc(-c2ccc3[nH]cnc3c2)cc1]

According to a preferred embodiment of the invention the molecule suitable to Formula IV is illustrated with Formula 80;

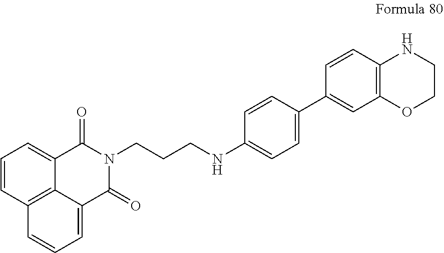 [SMILES: O=C1c2cccc3cccc(c23)C(=O)N1CCCNc1ccc(-c2ccc3c(c2)OCCN3)cc1]

According to a preferred embodiment of the invention the molecule suitable to Formula IV is illustrated with Formula 81;

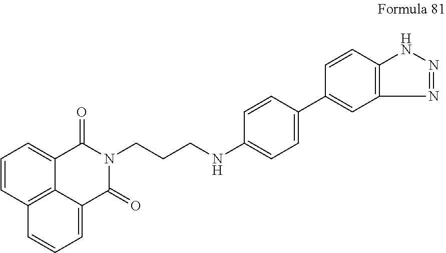 [SMILES: O=C1c2cccc3cccc(c23)C(=O)N1CCCNc1ccc(-c2ccc3[nH]nnc3c2)cc1]

According to a preferred embodiment of the invention the molecule suitable to Formula IV is illustrated with Formula 82;

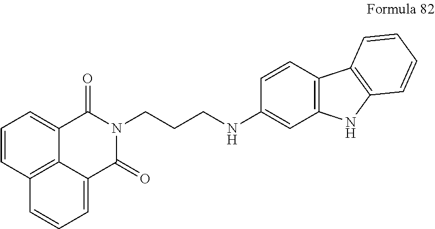 [SMILES: O=C1c2cccc3cccc(c23)C(=O)N1CCCNc1ccc2c(c1)[nH]c1ccccc12]

According to a preferred embodiment of the invention the molecule suitable to Formula IV is illustrated with Formula 83;

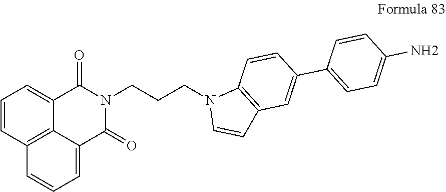 [SMILES: Nc1ccc(-c2ccc3c(ccn3CCCN3C(=O)c4cccc5cccc(c45)C3=O)c2)cc1]

According to a preferred embodiment of the invention the molecule suitable to Formula IV is illustrated with Formula 84;

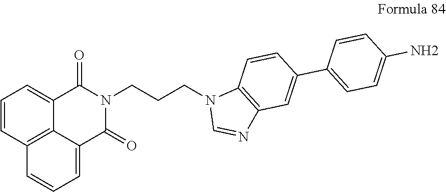 [SMILES: Nc1ccc(-c2ccc3c(c2)ncn3CCCN2C(=O)c3cccc4cccc(c34)C2=O)cc1]

According to a preferred embodiment of the invention the molecule suitable to Formula IV is illustrated with Formula 85;

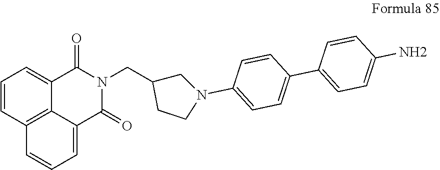 [SMILES: Nc1ccc(-c2ccc(N3CCC(CN4C(=O)c5cccc6cccc(c56)C4=O)C3)cc2)cc1]

According to a preferred embodiment of the invention the molecule suitable to Formula IV is illustrated with Formula 86;

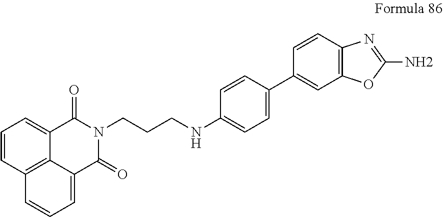 [SMILES: Nc1nc2ccc(-c3ccc(NCCCN4C(=O)c5cccc6cccc(c56)C4=O)cc3)cc2o1]

According to a preferred embodiment of the invention the molecule suitable to Formula IV is illustrated with Formula 87;

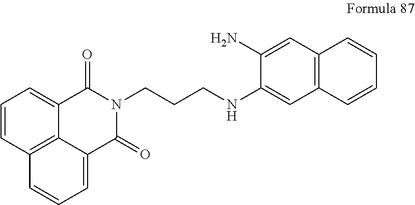 [SMILES: Nc1cc2ccccc2cc1NCCCN1C(=O)c2cccc3cccc(c23)C1=O]

According to a preferred embodiment of the invention the molecule suitable to Formula IV is illustrated with Formula 88;

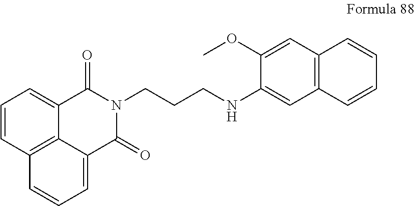 [SMILES: COc1cc2ccccc2cc1NCCCN1C(=O)c2cccc3cccc(c23)C1=O]

According to a preferred embodiment of the invention the molecule suitable to Formula IV is illustrated with Formula 89;

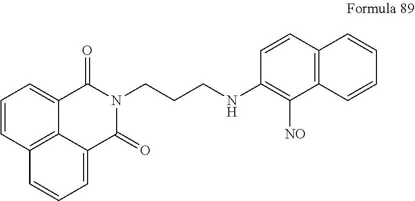

According to a preferred embodiment of the invention the molecule suitable to Formula IV is illustrated with Formula 90;

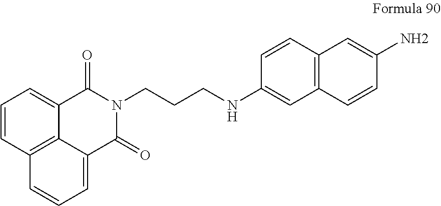 [SMILES: Nc1ccc2cc(NCCCN3C(=O)c4cccc5cccc(c45)C3=O)ccc2c1]

According to a preferred embodiment of the invention the molecule suitable to Formula IV is illustrated with Formula 91;

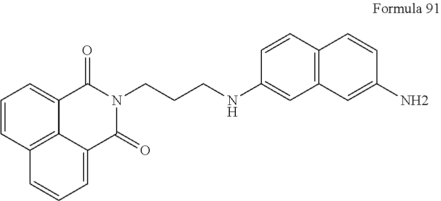 [SMILES: Nc1ccc2ccc(NCCCN3C(=O)c4cccc5cccc(c45)C3=O)cc2c1]

According to a preferred embodiment of the invention the molecule suitable to Formula IV is illustrated with Formula 92;

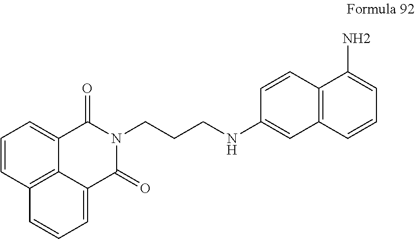 [SMILES: Nc1cccc2cc(NCCCN3C(=O)c4cccc5cccc(c45)C3=O)ccc12]

According to a preferred embodiment of the invention the molecule suitable to Formula IV is illustrated with Formula 93;

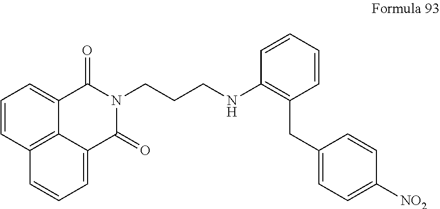 [SMILES: O=C1c2cccc3cccc(c23)C(=O)N1CCCNc1ccccc1Cc1ccc([N+](=O)[O-])cc1]

According to a preferred embodiment of the invention the molecule suitable to Formula IV is illustrated with Formula 94;

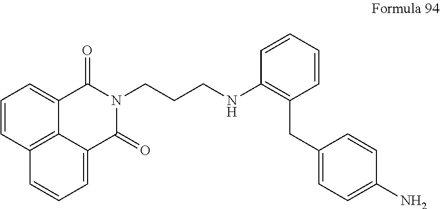 [SMILES: Nc1ccc(Cc2ccccc2NCCCN2C(=O)c3cccc4cccc(c34)C2=O)cc1]

According to a preferred embodiment of the invention the molecule suitable to Formula VII is illustrated with Formula 95;

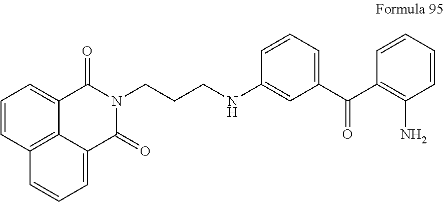 [SMILES: Nc1ccccc1C(=O)c1cccc(NCCCN2C(=O)c3cccc4cccc(c34)C2=O)c1]

According to a preferred embodiment of the invention the molecule suitable to Formula VII is illustrated with Formula 96;

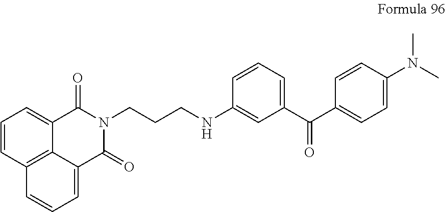 [SMILES: CN(C)c1ccc(C(=O)c2cccc(NCCCN3C(=O)c4cccc5cccc(c45)C3=O)c2)cc1]

According to a preferred embodiment of the invention the molecule suitable to Formula VII is illustrated with Formula 97;

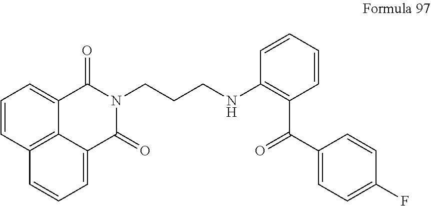 [SMILES: O=C(c1ccc(F)cc1)c1ccccc1NCCCN1C(=O)c2cccc3cccc(c23)C1=O]

According to a preferred embodiment of the invention the molecule suitable to Formula VII is illustrated with Formula 98;

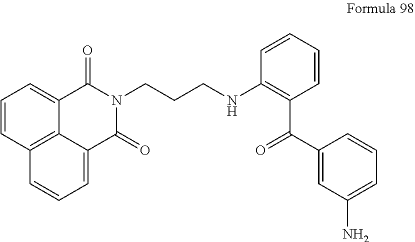 [SMILES: Nc1cccc(C(=O)c2ccccc2NCCCN2C(=O)c3cccc4cccc(c34)C2=O)c1]

According to a preferred embodiment of the invention the molecule suitable to Formula VII is illustrated with Formula 99;

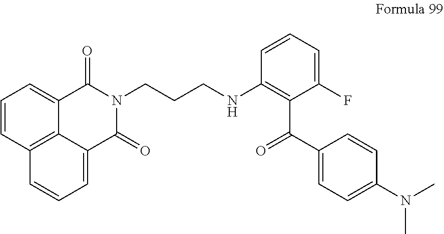 [SMILES: CN(C)c1ccc(C(=O)c2c(F)cccc2NCCCN2C(=O)c3cccc4cccc(c34)C2=O)cc1]

The molecules suitable to the invention shown with formulas 1-99 whose chemical structures and specific chemical names have been given above, have been provided to further describe the invention, wherein the scope of the invention is not limited to these molecules.

The method that can be used in obtaining the active agents shown with Formula I, Formula II, Formula III, Formula IV, Formula V, Formula VI, Formula VII, Formula VIII comprises the steps of;

a) Reacting the agent with paramethyl sulphonyl chloride in the presence of a solvent, preferably in the presence of dichloromethane as shown with Formula X,

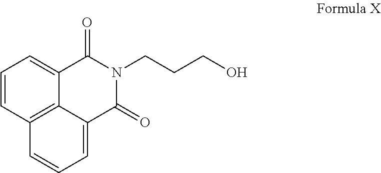 [SMILES: O=C1c2cccc3cccc(c23)C(=O)N1CCCO]

b) Mixing the obtained intermediary product (Formula Y) with NaI in the presence of a solvent, preferably acetonitrile and adding a diamino compound and K₂CO₃ solution in a solvent, preferably in acetonitrile

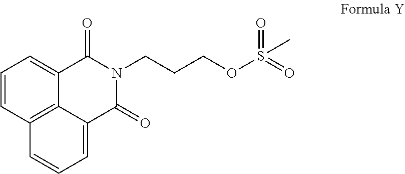 [SMILES: CS(=O)(=O)OCCCN1C(=O)c2cccc3cccc(c23)C1=O]

c) isolating the end product from the reaction mixture.

The term “solvent” as used herein, expresses any kind of organic solvent that can enable to dissolve reactive agents used during the reaction process.

Said organic solvent can be selected from the group formed of, acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, di ethylene glycol, diethylether, di ethylene glycol dimethyl ether, 1,2-dimethoxyethane (DME), dimethylformamide (DMF), dimethylsulphoxide (DMSO), 1,4-dioxane, ethanol, ethyl acetate, ethylene glycol, glycerine, heptane, hexamethylphosphoramide (HMPA), hexamethylphosphorus triamide (IMPT), hexane, methanol, methyl-butyl ether (MTBE), methylene chloride (DCM), N-methyl-yl-2-pyrrolidone (NMP), nitromethane, pentane, petrol ether, 1-propanol, 2-propanol, pyridine, tetrahydrofuran (THF), toluene, triethylamine, water, o-xylene, m-xylene, p-xylene.

The method illustrated with Method I, can be carried out at a temperature between the range of 0-90° C. or preferably at a temperature between the ranges of 25-80° C. Each step in the method can be carried out at a temperature that is different.

The products obtained by the methods according to the invention can be purified by methods such as filtration, centrifugation, extraction, crystallization, re-crystallization, chromatography and distillation.

The chromatography method mentioned here can be selected from column chromatography, gas chromatography, liquid chromatography, inverse phase liquid chromatography.

From another aspect, the invention described the usage of compounds illustrated with Formula I, Formula II, Formula III, Formula IV, Formula V, Formula VI, Formula VII, Formula VIII as a pharmaceutical product.

According to another aspect of the invention, the molecules illustrated with Formula I, Formula II, Formula III, Formula IV, Formula V, Formula VI, Formula VII, Formula VIII can be used in treating parasitic diseases.

The “parasitic diseases” term as used herein, represents diseases that are caused or infected by a parasite.

The parasitic diseases mentioned herein, can be, schistosomiasis, opisthorchiasis, clonorchiasis, dicrocoeliasis, fascioliasis, paragonimiasis, fasciolopsiasis, echinococcus, teniasis, cysticercosis, difilobotriasis, sparganosis, hymenolepiasis, dracunculiasis, onchocerciasis, filariasis, elephantiasis, mansonellosis, trichinellosis, ankilostomiasis, necatoriasis, ascaridosis, strongyloidiasis, trichuriosis, enterobiasis, anisakiasis, helmintiasis, trichostrongylus, angiostrongylus gnathostomiasis, angiostrongylus, syngamiasis, acanthocephaliasis, gongylonemiasis, metastrongylosis, thelaziasis, pediculosis, pityriasis, scabies, myiasis, tungiasis, trombiculosis, scrabiasis, hirudiniasis, linguatulosis, porocephaliasis, leishmaniasis, trypanosomiasis, malaria caused by Plasmodium falciparum, malaria caused by Plasmodium vivax, malaria caused by Plasmodium malariae, chagas disease, toxoplasma, pneumocystosis, babesiosis, piroplasmosis, acanthamoebiasis, naegleriasis and/or microsporidiosis.

According to a preferred embodiment of the invention, the molecules illustrated with Formula I, Formula II, Formula III, Formula IV, Formula V, Formula VI, Formula VII, Formula VIII can be used in a drug suitable to be used in treating leishmaniasis.

The invention preferably is related to treating leishmaniasis with the molecules illustrated with Formula I, Formula II, Formula III, Formula IV, Formula V, Formula VI, Formula VII, Formula VIII. The leishmaniasis disease mentioned here can be cutaneous, mucocutaneous or visceral leishmaniasis disease.

From another aspect the invention is related to the usage of the molecules shown with Formula I, Formula II, Formula III, Formula IV, Formula V, Formula VI, Formula VII, and Formula VIII to treat infectional diseases caused by parasites such as L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, L. venezuelensis.

According to another aspect of the invention, the molecules illustrated with Formula I, Formula II, Formula III, Formula IV, Formula V, Formula VI, Formula VII, Formula VIII can be used in treating bacterial diseases.

The “bacterial diseases” term as used herein, represents diseases arising from infections that are caused by pathogenic bacteria.

The bacterial diseases within the scope of the invention can be any bacterial disease formed of bacteria belonging to the main group of bacilli, Bartonella, bordetella, borrelia, brucella, campylobacter, chlamydia, chlamydophila, clostridium, corynebacterium, enterococcus, eccheria, Francisella, Haemophilus, helicobacter, legionella, leptospira, listeria, mycobacterium, mycoplasma, Neisseria, pseudomonas, rickettsia, salmonella, shigella, staphylococcus, streptococcus, treponema, ureaplasma, vibrio, yersinia.

Said bacterial diseases can be bubonic fever, pneumonic fever, cholera, syphilis, congenital syphilis, post streptotoxicglomerulonephritis, puerperal fever, impetigo, erysipelas, rheumatic fever, scarlet fever, streptococcall pharyngitis, sepsis, acute bacterial pneumonia, menengitis, otitis media, endometritis, neonatal sepsis, neonatal menengitis, neonatal pneumonia, cystitis, osteomyelitis, toxic shock syndrome, staphylococcal food poisoning, shigellose, salmonella, paratyphoid, typhoid, rocky mountain spotted fever, urinary tract infections, cornea infections, pneumonia, endocarditis, menengitis, gonorrhea, pelvic inflammatory disease, tuberculosis, leprosy, listeriosis, leptospirosis, legionary disease, peptic ulcer, upper respiratory tract infections, chronic gastritis, bronchitis, septic arthritis, tularemia, bloody diarrhea, urinary tract infections, endocarditis, biliary tract infections, diphtheria, tetanus, parrot disease, trachoma, neonatal conjunctivitis, urethritis, epididymitis, prostatitis, lymphogranulomatosis, atypical pneumonia, Guillain-Barre Syndrome, brucellosis, Lyme disease.

According to another aspect of the invention, the molecules illustrated with Formula I, Formula II, Formula III, Formula IV, Formula V, Formula VI, Formula VII, Formula VIII can be used in treating viral diseases.

The term “viral diseases” used within the scope of the present invention defines all of the diseases formed by the entrance of pathogenic viruses and infective virus particles (virions) into cells after bonding themselves to cells.

The viral diseases mentioned can be a disease caused by any kind of virus belonging to the main families of adenoviridae, herpesviridae, papillomaviridae, polyomaviridae, poxviridae, heradnaviridae, parvoviridae, astroviridae, caliciviridae, picornaviridae, coronaviridae, flaviviridae, togaviridae, hepeviridae, retroviridae, ortomiksoviridae, arenaviridae, bunyaviridae, filoviridae, paramiksoviridae, rabdoviridae, reoviridae.

Specific examples belonging to said viral diseases can be gastroenteritis, keratoconjunctivitis, pharyngitis, croup, pneumonia, cystitis, foot and mouth disease, pleurodnia, septic meningitis, pericarditis, myocarditis, Burkitt's Lymphoma, Hodgkin's lymphoma, nasopharyngeal carcinoma, acute hepatitis, chronic hepatitis, hepatic cirrhosis, hepatocellular carcinoma, tonsillitis, cytomegalic inclusion disease, Kaposi's sarcoma, Castleman disease, influenza, measles, Reye's syndrome, mumps, anogenital warts, poliomyelitis, rabies, congenital rubella, rubella, smallpox, zoster, congenital smallpox.

According to another aspect of the invention, the molecules illustrated with Formula I, Formula II, Formula III, Formula IV, Formula V, Formula VI, Formula VII, Formula VIII can be used in treating neoplastic diseases.

The term “neoplastic diseases” used within the scope of the invention related to a physiological condition, for example, cancer, which is characterized by malign tumors or uncontrolled cell growth. The terms “neoplastic diseases” and “cancer” can be used interchangeably with each other. Cancer examples although not limited to these, comprise carcinomas, lymphomas, blastoma sarcomas, and leukemia.

Carcinoma, as used herein, expresses a type of cancer formed of epithelium cells.

Lymphoma, as used herein, describes a cancer type developed from lymphocytes.

Blastoma, as used herein, describes a cancer type progressing from precursor cells also known as blast cells.

Sarcoma, as used herein, refers to the type of cancer resulting from altered cells of mesenchymal origin.

Leukemia, as used herein, expresses the type of cancer that is induced in the bone marrow and causes the formation of the high number of abnormal white blood cells.

Specific examples related to cancer types comprises breast cancer, prostate cancer, colorectal cancer, skin cancer, small cell lung cancer, non-small cell lung cancer, mesothelioma, gastrointestinal cancer, pancreatic cancer, glioblastoma, vulva cancer, cervical cancer, endometrial carcinoma, ovarian cancer, liver cancer, hepatoma, bladder cancer, kidney cancer, salivary gland carcinoma, thyroid cancer and various head and neck cancers.

The invention is furthermore related to a pharmaceutical composition comprising the compounds illustrated with Formula I, Formula II, Formula III, Formula IV, Formula V, Formula VI, Formula VII, Formula VIII suitable to the invention.

Said pharmaceutical compositions can comprise at least another active agent in addition to the compounds illustrated with Formula I, Formula II, Formula III, Formula IV, Formula V, Formula VI, Formula VII, Formula VIII.

According to another embodiment of the invention the compounds of the invention shown with Formula I, Formula II, Formula III, Formula IV, Formula V, Formula VI, Formula VII, Formula VIII can be used together with other active agents which have antiparasitic, antibacterial, antiviral, antineoplastic and/or cytotoxic and/or antimetastatic effects or with combinations comprising two/three of these agents. The second active agent can not only be formulated together with the compounds illustrated with Formula I, Formula II, Formula III, Formula IV, Formula V, Formula VI, Formula VII, Formula VIII, but it can also be sold in packages suitable to be used together after being formulated separately from the compounds suitable to Formula I, Formula II, Formula III, Formula IV, Formula V, Formula VI, Formula VII, Formula VIII.

According to another preferred embodiment of the invention, the compounds illustrated with Formula I, Formula II, Formula III, Formula IV, Formula V, Formula VI, Formula VII, Formula VIII is combined with at least one active agent which has antibacterial effects.

The active compounds which have antibacterial effects that can be used in combination with the compounds illustrated in Formula I, Formula II, Formula III, Formula IV, Formula V, Formula VI, Formula VII, Formula VIII, are selected from the group of amikacin, gentamicin, kanamycin, neomycin, netilmicin, tobramycin, paromycin, streptomycin, spectinomycin, geldanamycin, herbimycin, rifaximin, lorcarbef, ertapenem, doripenem, imipenem, meropenem, cefadroxil, cefazolin, cefalotin, cefalexin, cefaclor, cefamandol, cefoxitin, cefprozil, cefuroxime, cefixime, cefdinir, cefditoren, cefoperazone, cefotaxime, cefpodoxime, ceftazidime, ceftibutene, ceftizoxime, cefepime, ceftaroline fosamil, ceftobiprole, teicoplanin, vancomycine, televancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, meticillin, nafcillin, oxacillin, penicillin G, penicillin V, piperacillin, temocillin, ticarcillin, clavulanate, sulbactam, tazobactam, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, sulfadimethoxine, sulfamethizole, sulfamethoxazole, sulfanilamide, sulfasalazine, sulfisoxazole, trimethoprim, demeclocycline, doxycycline, minocycline, oxytetracycline, tetracycline, clofazimine, dapsone, capreomycin, cycloserine, ethambutol, isoniazid, pyrazinamide, rifampicin, rifabutin, rifapentine, streptomycin, arsphenamine, chloramphenicol, fosfomycin, fucidic acid, metronidazole, mupirocin, platensimycin, quinupristin, dalfopristin, thiamphenicol, tigecycline, tinidazole, trimethoprim or combinations comprising two or three of said agents.

According to a preferred embodiment of the invention, the compounds illustrated with Formula I, Formula II, Formula III, Formula IV, Formula V, Formula VI, Formula VII, Formula VIII is combined with at least one active agent which has antiviral effects.

The active agents that have antiviral effects, which can be used in combination with the compounds illustrated with Formula I, Formula II, Formula III, Formula IV, Formula V, Formula VI, Formula VII, Formula VIII can be selected from the group comprising abakacir, acyclovir, adefovir, amantadine, amprenavir, ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliver, famsiklovir, fomivirsen, fosamprenavir, foscarnet, fosfonate, ganciclovir, ibacitabine, imunovir, idoxuridine, imiquimod, indinavir, inosine, interpherone type I, interpherone type II, interpherone type III, interpherone, lamivudine, lopinavir, loviride, maraviroc, moroxydine, methisazone, nelfinavir, nevirapine, nexavir, nitazoxanide, norvir, oseltamivir, peginterferon alfa-2a, penciclovir, peramivir, pleconaril, podofilotoksin, protease inhibitor, nucleocyte analogues, raltegravir, ribavirin, rimantadine, ritonavir, pyramidine, saquinavir, sofosbuvir, stavudine, telaprevir, tenofovir, tipranavir, trifluridine, trizivir, tromantadine, truvada, valaciclovir, valganciclovir, vidarabine, viramidine, zalcitabine, zanamivir, zidovudine or from combinations comprising two or three of said agents.

According to a preferred embodiment of the invention, the compounds illustrated with Formula I, Formula II, Formula III, Formula IV, Formula V, Formula VI, Formula VII, Formula VIII is combined with at least one active agent which has antiparasitic effects.

Active compounds that have antiparasitic effect which can be used in combination with compounds illustrated with Formula I, can be selected from the group of, Nitazoxanide, melarsoprol, eflornithine, metronidazole, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, nitazoxanide, ornidazole, paromomycin sulphate, pentamidine, pyrimethamine, tinidazole, albendazole, mebendazole, thiabendazole, fenbendazole, triclabendazole, flubendazole, abamectin, diethylcarbamazine, ivermectin, suramin, pyrantel pamoate, levamisole, niclosamide, nitazoxanide, oxyclonazide, monepantel, derquantel, amphotericin B, urea stibamine, sodIum stibogluconate, meglumine antimoniate, paromomycin, miltefosine, fluconazole, pentamidine or combinations comprising two or three of said agents.

According to a preferred embodiment of the invention, the compounds illustrated with Formula I, Formula II, Formula III, Formula IV, Formula V, Formula VI, Formula VII, Formula VIII is combined with at least one active agent which has antineoplastic effects.

The active compounds which provide said antineoplastic effects that can be used in combination with the compounds illustrated with Formula I, Formula II, Formula III, Formula IV, Formula V, Formula VI, Formula VII, Formula VIII, can be selected from the group comprised of cyclophosphamide, iphosphamide, temozolomide, kapecitabin, 5-fluoro uracil, methotrexate, gemcitabine, pemetrexed, mitomycin, bleomycin, epirubicin, doxorubicin, etoposide, paclitaxel, irinotecan, osdocetaxel, vincristin, opcarboplatin, cisplatin, ocaliplatin, setcabecab, dibabecab, dibabec, rituximab, sunitinib, zoledronate, abiraterone, anastrozole, bicalutamide, exemestane, goserelin, medroxyprogesterone, octreotide, tamoxifen, bendamustine, carmustine, chlorambucil, lomustine, melphalan, procarbazine, streptozotocin fludarabine, raltitrexed, actinomycin D, dactinomycin, doxorubycin, mitoxantrone, erybulin, topotecan, vinblastine, vinorelbine, afatinib, aflibersept, chrysotinib, dabrafenib, interferon, ipilimumab, lapatinib, nivolumab, panitumumab, pembrolizumab, pertuzumab, sorafenib, trastuzumab, emtansin, temsoril, vemurafenib, ibandronic acid, pamidronate, bexarotane, buserelin, Cyproterone, degareliks, folinic acid, fulvestrant, lanreotide, lenalidomide, letrozole, leuprorelin, megestrol, mesna, thalidomide or combinations comprising two or three of said agents.

According to another embodiment of the invention at least another active agent can be formulized together with or separately from the compounds illustrated with Formula I, Formula II, Formula III, Formula IV, Formula V, Formula VI, Formula VII, Formula VIII, and at least said other active agents can have the same dosage as, or can have a different dosage from the compounds of Formula I, Formula II, Formula III, Formula IV, Formula V, Formula VI, Formula VII, Formula VIII.

In the case that at least another active agent mentioned above with the compounds of Formula I, Formula II, Formula III, Formula IV, Formula V, Formula VI, Formula VII, Formula VIII, is used, the other active agents, can be given at the same time with, successively with, or sequentially with the agents of Formula I, Formula II, Formula III, Formula IV, Formula V, Formula VI, Formula VII, Formula VIII.

The formulations according to the invention can comprise at least an excipient in addition to the active agents shown with Formula I, Formula II, Formula III, Formula IV, Formula V, Formula VI, Formula VII, Formula VIII.

The dosage range of the active agents that can be used together with Formula I, Formula II, Formula III, Formula IV, Formula V, Formula VI, Formula VII, Formula VIII shall be determined according to the requirements of the patient, the phase of the disease and the active agent to be used. The determination of a dose according to a specific situation is known by those that are skilled in the art.

The expression “comprising” within the scope of the invention is also used to mean “encompassing”.

Where it is technically suitable, the embodiments of the invention can be combined.

The embodiments have been described, such that they comprise the features/elements mentioned herein. The description essentially encompasses embodiments that comprise the mentioned features/elements or other embodiments constituted from these features/elements.

The patents and applications have been included in these documents by similar technical references.

The clearly described specific embodiments can constitute a basis to waiver singularly or together with a few embodiments.

The invention will be further described below by referring to the examples that have been provided only for illustration and which should not be construed to limit the scope of the invention.

### EXAMPLES

### Example 1: 2-(3-hydroxypropyl)-1H-benzo[de]isoquinoline-1,3(2H)-dione (Formula X) Synthesis Method

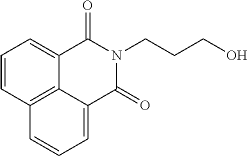 [SMILES: O=C1c2cccc3cccc(c23)C(=O)N1CCCO]

3-Amino-1-propanol (1 mmol) and DBU (1 mmol) is added into 4-amino-1,8-naphthalic anhydrite (1 mmol) solution dissolved in DMF and it is enabled to be mixed for 4 hours at 70° C. Following this iced water is added into the medium, and the solid established is filtered and a pure product is obtained. The progression of the reaction is checked with a DCM/MeOH:40/1 solvent system by conducting a TLC study. (Yield: %98) Analysis Data: LC-MS: m/z 256 [M+1]

### Example 2: 3-(1,3-dioxo-1H-benzo[de]isoquinoline-2(3H)-il)propyl methanesulfonate (Formula Y) Synthesis Method

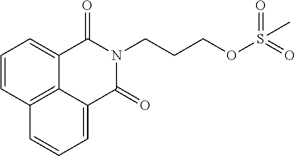 [SMILES: CS(=O)(=O)OCCCN1C(=O)c2cccc3cccc(c23)C1=O]

2-(3-hydroxypropyl)-1H-benzo[de]isoquinoline-1,3(2H)-dione is dissolved in DCM.

Following this TEA is added to the medium (1.2 mmol) and it was mixed for 15 minutes at 0° C. Following this para-methyl sulphonyl chloride (1.2 mmol) has been added into the medium and the reaction has been mixed for 2 hours. The progression of the reaction is checked with a DCM/MeOH (10/1) solvent. The reaction process was stopped with precipitation in ice water and filtering, and a pure product was obtained. (Yield: %86) Analysis Data: LC-MS: m/z 334 [M+1]

### Example 3: 2,2′-(((methylenebis(4,1-phenylene))bis(azanediyl))bis-(propane-3,1-diil))bis(1H-benzo[de]isoquinoline-1,3(2H)-dion) (C₄₃H₃₆N₄O₄) (Formula 1) Synthesis Method

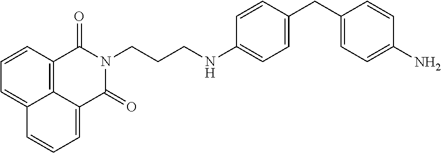 [SMILES: Nc1ccc(Cc2ccc(NCCCN3C(=O)c4cccc5cccc(c45)C3=O)cc2)cc1]

Formula Y (1 mmol) was mixed with NaI (6 mmol) for 2 hours at 70° C. in ACN medium. At the same time, it is enabled for Diaminodiphenylmethane (1 mmol) and K₂CO₃ (6 mmol) to be mixed in ACN medium at 70° C. for 2 hours in a reaction flask. Following this, these two reactions were mixed with each other and the reaction process was continued. TLC control was conducted and at the end of 12 hours, the reaction solvent was removed in a vacuum system. Following this extraction was carried out with aqueous NaCl solution and chloroform and the reaction was ended. The organic phase was placed inside dry Na₂SO₄ and was filtered and the solvent was removed. The purification process was carried out with column chromatography using a DCM/MeOH (40:1) solvent system during the purification of the product.

Analysis Data: LC-MS: m/z 436 [M+1].

¹H NMR (500 MHz, CDCl3): δ=1.95 (p, J=6.7 Hz, 2H, CH₂), 3.10 (t, J=6.5 Hz, 2H, NHCH₂), 3.66 (s, 2H, PhCH₂), 4.20 (t, J=6.9 Hz, 2H, NCH₂), 6.52-6.48 (m, 4H, 4×ArH), 6.87 (dd, J=8.2, 5.3 Hz, 4H, 4×ArH), 7.66-7.61 (m, 2H, 2×ArH), 8.09 (dd, J=8.3, 0.7 Hz, 2H, 2×ArH), 8.48 (dd, J=7.3, 0.9 Hz, 2H, 2×ArH) ppm.

### Example 4: 2,2′-(([1,1′-biphenyl]-4,4′-diylbis(azanediyl))bis-(propane-3,1-diil))bis(1H-benzo[de]isoquinoline-1,3(2H)-dion), (C₄₂H₃₄N₄O₄) (Formula 9) Synthesis Method

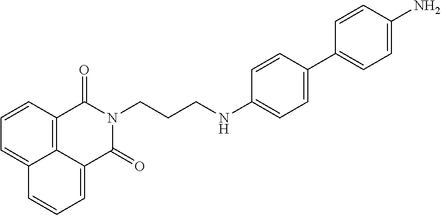 [SMILES: Nc1ccc(-c2ccc(NCCCN3C(=O)c4cccc5cccc(c45)C3=O)cc2)cc1]

Formula Y (1 mmol) was mixed with NaI (6 mmol) for 2 hours at 70° C. in ACN medium. At the same time, it is enabled for Benzidine (1 mmol) and K₂CO₃ (6 mmol) to be mixed in ACN medium at 70° C. for 2 hours in a reaction flask. Following this, these two reactions were mixed with each other and the reaction process was continued. TLC control was conducted and at the end of 12 hours, the reaction solvent was removed in a vacuum system. Following this extraction was carried out with aqueous NaCl solution and chloroform and the reaction was ended. The organic phase was placed inside dry Na₂SO₄ and was filtered and the solvent was removed. The purification process was carried out with column chromatography using a DCM/MeOH (20:1) solvent system during the purification of the product.

Analysis Data: LC-MS: m/z 423 [M+1].

¹H NMR (500 MHz, CDCl3): δ=1.99-2.06 (m, 2H, CH₂), 3.18 (t, J=6.4 Hz, 2H, NHCH₂), 4.26 (t, J=6.8 Hz, 2H, NCH₂), 6.67-6.61 (m, 4H, 4×ArH), 7.30-7.25 (m, 2H, 4×ArH), 7.69 (dd, J=12.8, 5.2 Hz, 2H, 2×ArH), 8.16 (dd, J=8.2, 0.7 Hz, 2H, 2×ArH), 8.55 (dd, J=7.3, 0.9 Hz, 2H, 2×ArH) ppm.

### Example 5: 2-(3-((5-amino naphthalene-1-il)amino)propyl)-1H-benzo[de]isoquinoline-1,3(2H)-dione, (C₂₅H₂₁N₃O₂) (Formula 61) Synthesis Method

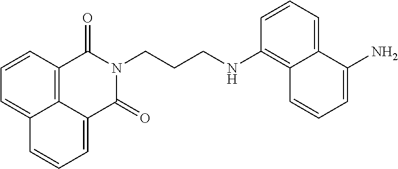 [SMILES: Nc1cccc2c(NCCCN3C(=O)c4cccc5cccc(c45)C3=O)cccc12]

Formula Y (1 mmol) was mixed with NaI (6 mmol) for 2 hours at 70° C. in ACN medium. At the same time, it is enabled for 1.5-Diaminodiphenylmethane (1 mmol) and K₂CO₃ (6 mmol) to be mixed in ACN medium at 70° C. for 2 hours in a reaction flask. Following this, these two reactions were mixed with each other and the reaction process was continued. TLC control was conducted and at the end of 12 hours, the reaction solvent was removed in a vacuum system. Following this extraction was carried out with aqueous NaCl solution and chloroform and the reaction was ended. The organic phase was placed inside dry Na₂SO₄ and was filtered and the solvent was removed. The purification process was carried out with column chromatography using a DCM/MeOH (25:1) solvent system during the purification of the product.

Analysis Data: LC-MS: m/z 396 [M+1].

¹H NMR (500 MHz, CDCl3): δ=2.13 (p, J=6.6 Hz, 2H, CH₂), 3.33 (t, J=6.3 Hz, 2H, NHCH₂), 4.33 (t, J=6.7 Hz, 2H, NCH₂), 6.57 (d, J=7.6 Hz, 1H, ArH), 6.71 (d, J=73 Hz, 1H, ArH), 7.09 (d, J=8.4 Hz, 1H, ArH), 7.22 (dd, J=8.8, 1.3 Hz, 1H, ArH), 7.28-7.24 (m, 1H, ArH), 7.41 (t, J=6.1 Hz, 1H, ArH), 7.70 (dt, J=11.6, 5.8 Hz, 2H, 2×ArH), 8.17 (dd, J 8.3, 0.9 Hz, 2H, 2×ArH), 8.58 (dd, J=7.3, 1.0 Hz, 2H, 2×ArH) ppm.

### Example 6: 2-(3-((8-amino naphthalene-1-yl)amino)propyl)-1H-benzo[de]isoquinoline-1,3(2H)-dione, (C₂₅H₂₁N₃O₂) (Formula 62) Synthesis Method

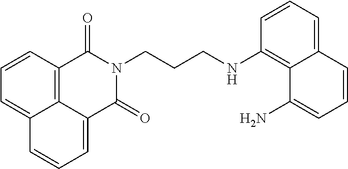 [SMILES: Nc1cccc2cccc(NCCCN3C(=O)c4cccc5cccc(c45)C3=O)c12]

Formula Y (1 mmol) was mixed with NaI (6 mmol) for 2 hours at 70° C. in ACN medium. At the same time, it is enabled for 1,8-Diaminodiphenylmethane (1 mmol) and K₂CO₃ (6 mmol) to be mixed in ACN medium at 70° C. for 2 hours in a reaction flask. Following this, these two reactions were mixed with each other and the reaction process was continued. TLC control was conducted and at the end of 12 hours, the reaction solvent was removed in a vacuum system. Following this extraction was carried out with aqueous NaCl solution and chloroform and the reaction was ended. The organic phase was placed inside dry Na₂SO₄ and was filtered and the solvent was removed. The purification process was carried out with column chromatography using a DCM/MeOH (100:1) solvent system during the purification of the product.

Analysis Data: LC-MS: m/z 396 [M+1].

¹H NMR (500 MHz, CDCl3): δ=2.17-2.13 (m, 2H, CH₂), 3.19 (t, J=6.5 Hz, 2H, NHCH₂), 4.34 (dd, J=13.9, 6.8 Hz, 2H, NCH₂), 6.57 (t, J=8.2 Hz, 1H), 6.64 (d, J=7.2 Hz, 1H), 7.11 (d, J=4.1 Hz, 1H), 7.13 (d, J=3.7 Hz, 1H, ArH), 7.18 (d, J=3.3 Hz, 1H, ArH), 7.20 (d, J 5.4 Hz, 1H, ArH), 7.71-7.67 (m, 2H, 2×ArH), 8.15 (dd, J=8.3, 1.0 Hz, 2H, 2×ArH), 8.54 (dd, J=7.3, 1.0 Hz, 2H, 2×ArH) ppm.

### Example 7: Biological Activity of Formula 9 Against Leishmania Infantum Parasites

Method and Materials:

Parasite Kulture: Leishmania infantum (MHOM/MA/67/ITMA-P263) promastigotes are incubated in an RPMI medium (with heat-activated 10% fetal bovine serum, 2 mM L-glutamine, 20 mM HEPES, 100 U/ml penicillin, 100 μg/ml streptomycin) at 27° C. The parasites that reach the logarithmic phase (10⁶/ml) become infective.

Alamar Blue Cell Viability Test: After the Leishmania infantum (MHOM/MA/67/ITMA-P263) promastigotes were added into RPMI medium (with heat-activated 10% fetal bovine serum, 2 mM L-glutamine, 20 mM HEPES, 100 U/ml penicillin, 100 μg/ml streptomycin) at 27° C., in 96 well culture plates (Corning Glasswork, Corning, N.Y.) such that they are 8×10⁶ parasite/wells, on the 3rd day the viability levels of parasites were measured. Alamar Blue test (Sigma-Aldrich, Germany) is applied to determine the viabilities of parasites. 10p Alamar Blue solution is added onto the cells inside the 100 μl growth medium and the cells are incubated for a day in the dark. Following the incubation period, absorbance measurement was taken with an ELISA plaque reader (Biotek, Winooski, Vt.) at 530/590 nm wavelength and viability analysis was observed.

Result:

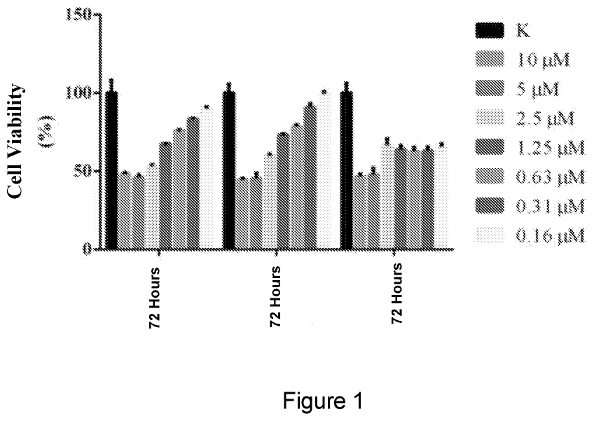

The effect of the molecule with Formula 9 defined within the scope of the invention, on Leishmania infantum parasites has been shown. The Formula 9 compound was added onto the parasites, and when viability was analyzed for 3 days, the death rate up to an average of 45% was observed (FIG. 1). Under the light of the assays carried out, it has been determined that the developed molecule was effective on Leishmania infantum parasites.

1. Bisnaphthalimidopropyl (BNIP) derived molecules, wherein said molecules are illustrated with; 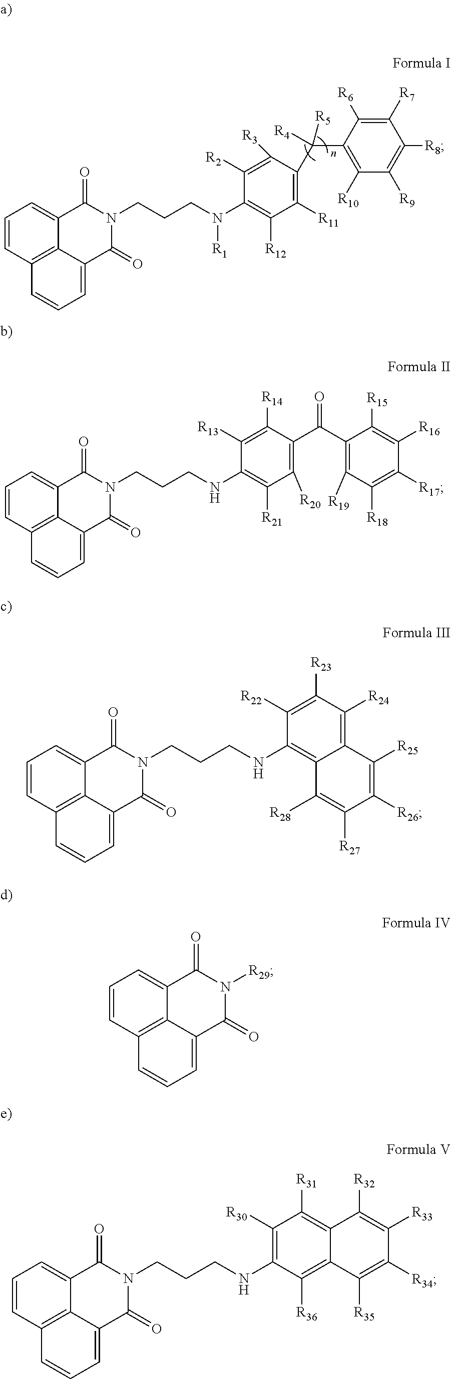 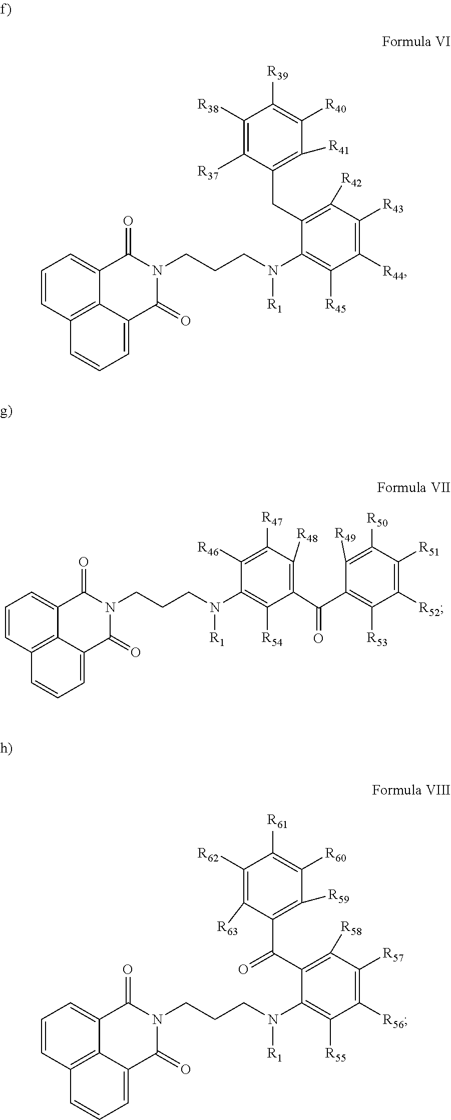
wherein;
-n is a natural number between 0 to 12,
—R₁, R₂, R₃, R₄, R₅, R₆, R₇, R₈, R₉, R₁₀, R₁₁, R₁₂, R₃₇, R₃₈, R₃₉, R₄₀, R₄₁, R₄₂, R₄₃, R₄₄, R₄₅, R₄₆, R₄₇, R₄₈, R₄₉, R₅₀, R₅₁, R₅₂, R₅₃, R₅₄, R₅₅, R₅₆, R₅₇, R₅₈, R₅₉, R₆₀, R₆₁, R₆₂, R₆₃, are independently selected from the group consisting of:
—H, —NH₂, —NHR_{X}, —NR_{X}R_{Y}, —NHNR_{X}, —N(R_{X})(NHR_{Y}), —N(R_{X})(NR_{Y}R_{Z}), R_{X}, R_{Y}, R_{Z}, Cl, F, Br, I, —OH, —NO, —NO₂, —COOH, —OR_{X}, —SO₂R_{X}, —SO₂NHR_{X}, —SO₂NR_{X}R_{Y}, —CN, —COOR_{X}, —CONHR_{X}, —CONR_{X}R_{Y}, —NHCOCH₂R_{X}, —NHCOCHR_{X}R_{Y}, —NHCNHR_{X}, —NHCOR_{X}, —NHCONHR_{X}, —NHSO₂R_{X};
—R₁₃, R₁₄, R₁₅, R₁₆, R₁₇, R₁₈, R₁₉, R₂₀, R₂₁ are independently selected from the group consisting of:
—H, —Cl, —F, —Br, —I, R_{X}, —NH₂, —NHR_{X}, —NR_{X}R_{Y}, —OH, —NO, —NO₂, —COOH, —OR_{X}, —COOR_{X}, —CN;
—R₂₂, R₂₃, R₂₄, R₂₅, R₂₆, R₂₇, R₂₈ are independently selected from the group consisting of:
—H, —Cl, —F, —Br, —I, R_{X}, —NH₂, —NHR_{X}, —NR_{X}R_{Y}, —OH, —COOH, —OR_{X}, —COOR_{X}, —CN, —SO₃H, —SO₃ Na⁺, 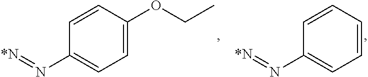
—NO;
—R₂₉ is selected from the group consisting of: 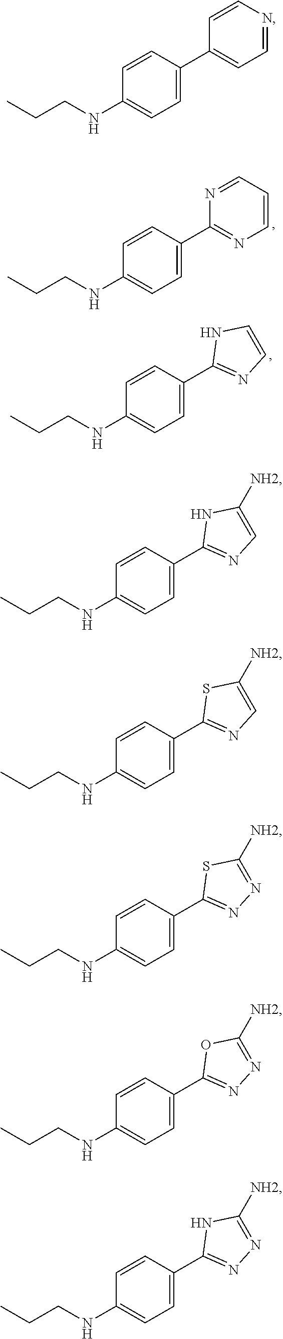 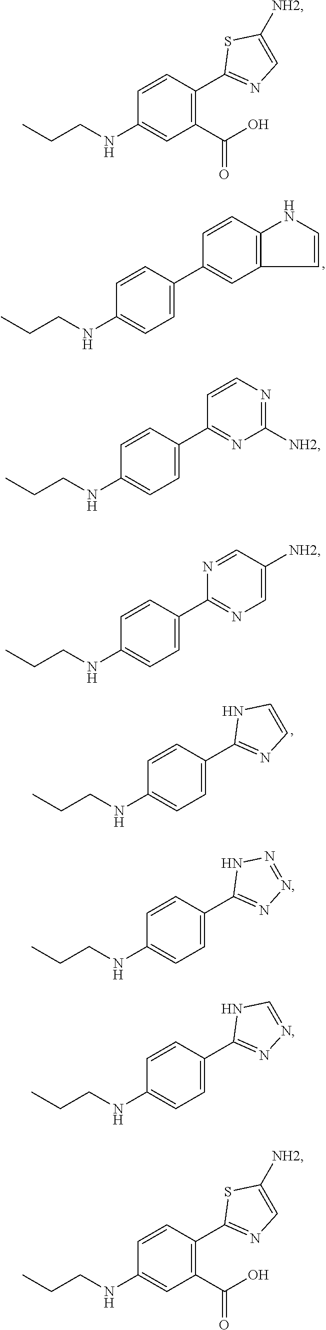 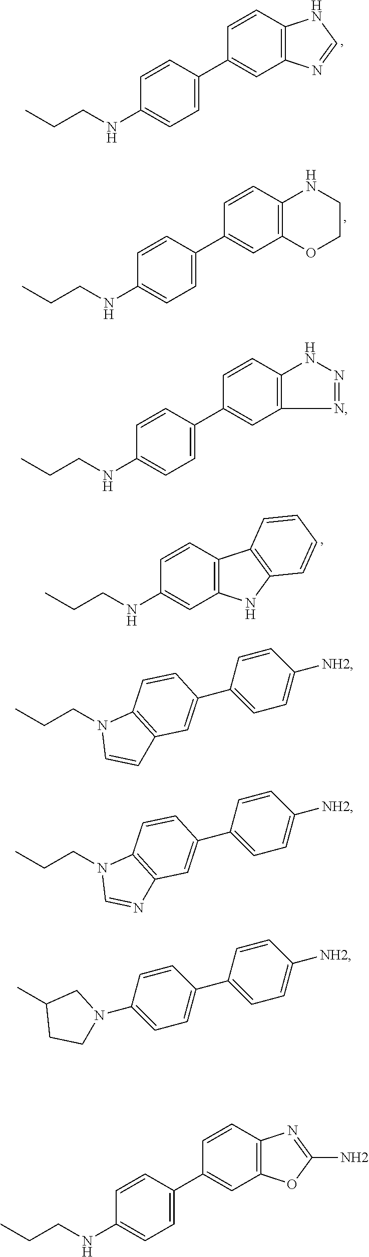
—R₃₀, R₃₁, R₃₂, R₃₃, R₃₄, R₃₅, R₃₆ are independently selected from the group consisting of:
—H, —Cl, —F, —Br, —I, R_{X}, —NH₂, —NHR_{X}, —NR_{X}R_{Y}, —OH, —NO, —COOH, —OR_{X}, —COOR_{X}, —CN;
R_{X}, R_{Y}, and R_{Z} are independently selected from substituted or unsubstituted C1-C12 alkane, substituted or unsubstituted C1-C12 alkene, substituted or unsubstituted C1-C12 alkyne, substituted or unsubstituted C1-C12 primary amine, substituted or unsubstituted C1-C12 secondary amine, substituted or unsubstituted alcohol, substituted or unsubstituted alkyl cyanide, substituted or unsubstituted sulphonamide, substituted or unsubstituted C1-C12 alkyl sulphonyl, substituted or unsubstituted C3-C8 cycloalkyl, substituted or unsubstituted aryl, substituted or unsubstituted heteroaryl, optionally and independent from each other from a group formed of substituted or unsubstituted heterocyclic or heteroaromatic rings that comprise one or more heteroatoms selected from O, S and N.

2. Bisnaphthalimidopropyl (BNIP) derived molecules according to claim 1, wherein n takes the value of 0 or 1 or 2 or 3 or 4 or 5 or 6 or 7 or 8 or 9 or 10 or 11 or 12 in the molecules illustrated with Formula I;
R₁, R₂, R₃, R₄, R₅, R₆, R₇, R₈, R₉, R₁₀, R₁₁ ve R₁₂ are independently selected from the group consisting of:
—H, —NH₂, —CH₃, —CH₂CH₃, —CH₂CH₂CH₃, —CH₂CH₂CH₂CH₃, —CH₂CH₂CH₂CH₂CH₃, —CH₂CH₂CH₂CH₂CH₂CH₃, CH₂CH₂CH₂CH₂CH₂CH₂CH₃, —CH₂CH₂CH₂CH₂CH₂CH₂CH₂CH₃, —CH₂CH₂CH₂CH₂CH₂CH₂CH₂CH₂CH₃, CH₂CH₂CH₂CH₂CH₂CH₂CH₂CH₂CH₂CH₃, —CH₂CH₂CH₂CH₂CH₂CH₂CH₂CH₂CH₂CH₂CH₃, —CH₂CH₂CH₂CH₂CH₂CH₂CH₂CH₂CH₂CH₂CH₂CH₃, —C(CH₃)₃, Cl, F, Br, I, —OH, —OCH₃, —OCH₂CH₃, —OCH₂CH₂CH₃, —OCH₂CH₂CH₂CH₃, —OCH₂CH₂CH₂CH₂CH₃, —OCH₂CH₂CH₂CH₂CH₂CH₃, —NHCH₃, —NHCH₂CH₃, —NHCH₂CH₂CH₃, —NHCH₂CH₂CH₂CH₃, —NHCH₂CH₂CH₂CH₂CH₃, —NHCH₂CH₂CH₂CH₂CH₂CH₃, —N(CH₃)₂, —N(CH₂CH₃)(CH₃), —N(CH₂CH₃)₂, —N(CH₂CH₃)(CH₂CH₂CH₃), —N(CH₃)(CH₂CH₂CH₃), N(CH₂CH₂CH₃)₂, —NHNH₂, —NHNHCH₃, —NHNHCH₂CH₃, —NHNHCH₂CH₂CH₃, —NHN(CH₃)₂, —NHN(CH₂CH₃)₂, —NHN(CH₃)(CH₂CH₃), —N(CH₃)(NHCH₃), —N(CH₃)(N(CH₃)₂), —COOH, —SO₂CH₃, —SO₂CH₂CH₂, —SO₂CH₂CH₂CH₃, —SO₂CH₂CH₂CH₂CH₃, —SO₂CH₂CH₂CH₂CH₂CH₃, —SO₂CH₂CH₂CH₂CH₂CH₂CH₃, —CN, —CONH₂, —CONHCH₃, —CONHCH₂CH₃, —CONHCH₂CH₂CH₃, —CONHCH₂CH₂CH₂CH₃, —CONHCH₂CH₂CH₂CH₂CH₃, —CONHCH₂CH₂CH₂CH₂CH₂CH₃, —CON(CH₃)₂, —CON(CH₃)(CH₂CH₃), —CON(CH₂CH₃)₂, —CON(CH₂CH₃)(CH₂CH₂CH₃), —CON(CH₃)(CH₂CH₂CH₃), —CON(CH₂CH₂CH₃)₂, —COCH₃, —COCH₂CH₃, —COCH₂CH₂CH₃, —COCH₂CH₂CH₂CH₃, —COCH₂CH₂CH₂CH₂CH₃, —COCH₂CH₂CH₂CH₂CH₂CH₃, —COOCH₃, —COOCH₂CH₃, —COOCH₂CH₂CH₃, —COOCH₂CH₂CH₂CH₃, —COOCH₂CH₂CH₂CH₂CH₃, —COOCH₂CH₂CH₂CH₂CH₂CH₃, —COCH(CH₃)₂, —COC(CH₃)₃, —COCH(CH₂CH₃)₂, —COCH(CH₃)(CH₂CH₃), —COCH₂CH₂CH₃, —COCH₂CH₂CH₂CH₃, —COCH₂CH₂CH₂CH₂CH₃, —COCH₂CH₂CH₂CH₂CH₂CH₃, —SO₂NH₂, SO₂NHCH₃, SO₂NHCH₂CH₃, —SO₂NHCH₂CH₂CH₃, SO₂N(CH₃)₂, —SO₂N(CH₃)(CH₂CH₃), —SO₂N(CH₂CH₃)₂, —SO₂N(CH₃)(CH₂CH₂CH₃), —SO₂N(CH₂CH₂CH₃)₂, —NHCONHCH₃, —NHCON(CH₃)₂, —NHCONHCH₂CH₃, NHCON(CH₃)(CH₂CH₃), —NHCON(CH₂CH₃)₂, —NHCOCH(CH₃)(NH₂), —NHCOCH(CH₃)₂, —NHCOCH(CH₃)(NHCH₃), —NHCOCH(CH₃)(N(CH₃)₂), —NHCOCH(CH₂CH₃)₂, —NHCOCH(CH₂OH)(NH₂), —NHCOCH(CH₂CH₂OH)(NH₂), —NHCOCH(CH₂CH₂OH)(NHCH₃), —NHCOCH(CH₂OH)(NHCH₃), NHCOCH(CH₂OH)(N(CH₃)₂), NHCOCH(CH₂OH)(N(CH₂CH₃)₂), —NHCOCH(CH₂SH)(NH₂), —NHCOCH(CH₂CH₂SH)(NH₂), —NHCOCH(CH₂CH₂CH₂SH)(NH₂), —NHCOCH(CH₂SH)(NHCH₃), —NHCOCH(CH₂SH)(NHCH₂CH₃), —NHCOCH(CH₂SH)(N(CH₃)₂), 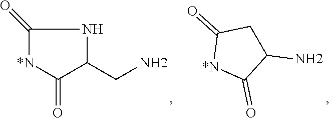
—NH(CNH)NH₂, —NH(CN(CH₃))NH₂, —NH(CN(CH₂CH₃))NH₂, NH(CN(CH₃))NH(CH₃), —NH(CN(CH₃))N(CH₃)₂, —NH(CNH)NHCH₃, NH(CNH)N(CH₃)₂, —NH(CO)NHCH₂CH₂NH₂, —NH(CO)NHCH₂NH₂, —NH(CO)NHCH₂CH₂NHCH₃, —NH(CO)N(CH₃)(CH₂CH₂NH₂), —NH(CO)CH₂CH₂NH₂, —NH(CO)CH₂NH₂, —NH(CO)CH₂CH₂CH₂NH₂, —NH(CO)CH₂CH₂CH₂CH₂NH₂, —NH(CO)CH₂CH₂NHCH₃, —NH(CO)CH₂CH₂N(CH₃)₂, —NH(CO)CH₂CH₂NH(CH₂CH₃), —NH(CO)CH₂CH₂N(CH₃)(CH₂CH₃), —NHSO₂CH₂CH₂NH₂, —NHSO₂CH₂CH₂CH₂NH₂, —NHSO₂CH₂CH₂NH₂HCH₃, —NHSO₂CH₂CH₂NH₂(CH₃)₂, —NHSO₂CH₂CH₂NH₂(CH₃)(CH₂CH₃), —NHSO₂CH₂CH₂NH₂(CH₂CH₃)(CH₂CH₃), —NHCH₂CH₂NH₂, —NHCH₂NH₂, —NHCH₂CH₂CH₂NH₂, —NHCH₂CH₂CH₂CH₂NH₂, —NHCH₂CH₂NHCH₃, —NHCH₂CH₂N(CH₃)₂, —N(CH₂CH₂NH₂)₂, —N(CH₂CH₂CH₂NH₂)₂, —N(CH₂NH₂)₂, —N(CH₂NHCH₃)₂, —N(CH₂CH₂NHCH₃)₂, —N(CH₂CH₂CH₂NHCH₃)₂, —NHCONHCH₂CH₂SO₂NH₂, —NHCONHCH₂CH₂CH₂SO₂NH₂, —NHCON(CH₃)(CH₂CH₂CH₂SO₂NH₂), —NHCON(CH₂CH₃)(CH₂CH₂CH₂SO₂NH₂), —NHCON(CH₃)(CH₂CH₂SO₂NH₂), —NHCONHCH₂CH₂SO₂NHCH₃, —NHCONHCH₂CH₂SO₂NHCH₂CH₃, —NHCONHCH₂CH₂SO₂N(CH₃)₂, —NHCH₂CH₂SO₂NH₂, —NHCH₂CH₂CH₂SO₂NH₂, —NHCH₂CH₂CH₂CH₂SO₂NH₂, NHCH₂CH₂SO₂NHCH₃, —NHCH₂CH₂SO₂N(CH₃)₂.

3. Bisnaphthalimidopropyl (BNIP) derived molecules according to claim 1, wherein in Formula I, when n=0, R₄, and R₅ are null.

4. Bisnaphthalimidopropyl (BNIP) derived molecules according to claim 1, wherein R₁₃, R₁₄, R₁₅, R₁₆, R₁₇, R₁₈, R₁₉, R₂₀, R₂₁ in the molecules illustrated with formula II are independently selected from the group consisting of —H, —Cl, —F, —Br, —I, —NH₂, —CH₃, C(CH₃)₂.

5. Bisnaphthalimidopropyl (BNIP) derived molecules according to claim 1, wherein R₃₀, R₃₁, R₃₂, R₃₃, R₃₄, R₃₅, R₃₆ in the molecules illustrated with formula V are independently selected from the group consisting of —H, —NH₂, —OCH₃, —NO, —Cl, F, I, Br.

6. Bisnaphthalimidopropyl (BNIP) derived molecules according to claim 1, wherein R₁, R₃₇, R₃₈, R₃₉, R₄₀, R₄₁, R₄₂, R₄₃, R₄₄, R₄₅ in the molecules illustrated with formula VI are independently selected from the group consisting of —H, —NH₂, —CH₃, —CH₂CH₃, —CH₂CH₂CH₃, —C(CH₃)₃, Cl, F, Br, I, —OH, —OCH₃, —OCH₂CH₃, —NHCH₃, —NHCH₂CH₃, —N(CH₃)₂, —NO₂, —COOH, —COOCH₃, —COONH₂, —COONHCH₃, —COON(CH₃)₂.

7. Bisnaphthalimidopropyl (BNIP) derived molecules according to claim 1, wherein R₁, R₄₆, R₄₇, R₄₈, R₄₉, R₅₀, R₅₁, R₅₂, R₅₃, R₅₄ in the molecules illustrated with formula VII are independently selected from the group consisting of —H, —NH₂, —CH₃, —CH₂CH₃, —CH₂CH₂CH₃, —C(CH₃)₃, Cl, F, Br, I, —OH, —OCH₃, —OCH₂CH₃, —NHCH₃, —NHCH₂CH₃, —N(CH₃)₂, —NO₂, —COOH, —COOCH₃, —COONH₂, —COONHCH₃, —COON(CH₃)₂.

8. Bisnaphthalimidopropyl (BNIP) derived molecules according to claim 1, wherein R₁, R₅₅, R₅₆, R₅₇, R₅₈, R₅₉, R₆₀, R₆₁, R₆₂, R₆₃ in the molecules illustrated with formula VIII are independently selected from the consisting group of —H, —NH₂, —CH₃, —CH₂CH₃, —CH₂CH₂CH₃, —C(CH₃)₃, Cl, F, Br, I, —OH, —OCH₃, —OCH₂CH₃, —NHCH₃, —NHCH₂CH₃, —N(CH₃)₂, —NO₂, —COOH, —COOCH₃, —COONH₂, —COONHCH₃, —COON(CH₃)₂.

9. Bisnaphthalimidopropyl (BNIP) derived molecules according to claim 6, wherein in molecules illustrated with Formula VI, VII, and VIII, R₁ is —H.

10-108. (canceled)

109. Method for use in the preparation of bisnaphthalimidopropyl (BNIP) derivative molecules according to claim 1;
a) Reacting the agent shown with Formula X with paramethyl sulphonyl chloride in the presence of a solvent, 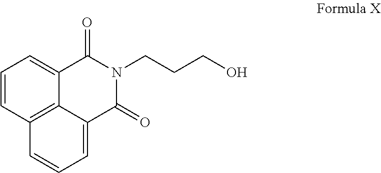 [SMILES: O=C1c2cccc3cccc(c23)C(=O)N1CCCO]
b) Mixing the obtained intermediary product (Formula Y) with NaI in the presence of a solvent and adding a diamino compound and K₂CO₃ solution in a solvent, preferably in acetonitrile 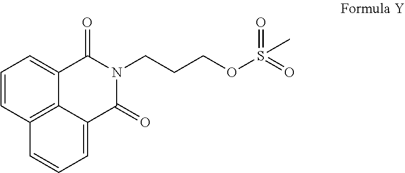 [SMILES: CS(=O)(=O)OCCCN1C(=O)c2cccc3cccc(c23)C1=O]
c) isolating the end product from the reaction mixture.

110-112. (canceled)

113. Bisnaphthalimidopropyl (BNIP) derivative molecules shown with Formula I, Formula II, Formula III, Formula IV, Formula V, Formula VI, Formula VII, Formula VIII according to claim 1 for use as a drug for treatment of bacterial, viral, parasitic and neoplastic diseases.

114. Bisnaphthalimidopropyl (BNIP) derivative molecules according to claim 113, wherein the parasitic disease, can be, schistosomiasis, opisthorchiasis, clonorchiasis, dicrocoeliasis, fascioliasis, paragonimiasis, fasciolopsiasis, echinococcus, teniasis, cysticercosis, difilobotriasis, sparganosis, hymenolepiasis, dracunculiasis, onchocerciasis, filariasis, elephantiasis, mansonellosis, trichinellosis, ankilostomiasis, necatoriasis, ankilostomiasis, ascaridosis, strongyloidiasis, trichuriosis, enterobiasis, anisakiasis, helmintiasis, trichostrongylus, angiostrongylus gnathostomiasis, angiostrongylus, syngamiasis, hirudiniasis, acanthocephaliasis, gongylonemiasis, metastrongylosis, thelaziasis, pediculosis, pityriasis, scabies, myiasis, tungiasis, trombiculosis, scrabiasis, hirudiniasis, linguatulosis, porocephaliasis, leishmaniasis, trypanosomiasis, malaria caused by Plasmodium falciparum, malaria caused by Plasmodium vivax, malaria caused by Plasmodium malariae, chagas disease, toxoplasma, pneumocystosis, babesiosis, piroplasmosis, acanthamoebiasis, naegleriasis and/or microsporidiosis.

115-117. (canceled)

118. Bisnaphthalimidopropyl (BNIP) derivative molecules according to claim 113 wherein the bacterial disease can be bubonic fever, pneumonic fever, cholera, syphilis, congenital syphilis, post streptococcic glomerulonephritis, puerperal fever, impetigo, erysipelas, rheumatic fever, scarlet fever, streptococcal pharyngitis, sepsis, acute bacterial pneumonia, meningitis, otitis media, endometritis, neonatal sepsis, neonatal meningitis, neonatal pneumonia, cystitis, osteomyelitis, toxic shock syndrome, staphylococcal food poisoning, shigellosis, salmonella, paratyphoid, typhoid, rocky mountain spotted fever, urinary tract infections, cornea infections, pneumonia, endocarditis, meningitis, gonorrhea, pelvic inflammatory disease, tuberculosis, leprosy, listeriosis, leptospirosis, legionary disease, peptic ulcer, upper respiratory tract infections, chronic gastritis, bronchitis, septic arthritis, tularemia, bloody diarrhea, urinary tract infections, endocarditis, biliary tract infections, diphtheria, tetanus, parrot disease, trachoma, neonatal conjunctivitis, urethritis, epididymitis, prostatitis, lymphogranulomatosis, atypical pneumonia, Guillain-Barre Syndrome, brucellosis, Lyme disease.

119. (canceled)

120. Bisnaphthalimidopropyl (BNIP) derivative molecules according to claim 113 wherein the viral disease can be gastroenteritis, keratoconjunctivitis, pharyngitis, croup, pneumonia, cystitis, foot and mouth disease, pleurodynia, septic meningitis, pericarditis, myocarditis, Burkitt's Lymphoma, Hodgkin's lymphoma, nasopharyngeal carcinoma, acute hepatitis, chronic hepatitis, hepatic cirrhosis, hepatocellular carcinoma, tonsillitis, cytomegalic inclusion disease, Kaposi's sarcoma, Castleman disease, influenza, measles, Reye's syndrome, mumps, anogenital warts, poliomyelitis, rabies, congenital rubella, rubella, smallpox, zoster, congenital smallpox.

121. Bisnaphthalimidopropyl (BNIP) derivative molecules according to claim 113, wherein the neoplastic disease can be cancer, such as carcinoma, lymphoma, blastoma, sarcoma, leukemia, breast cancer, prostate cancer, colorectal cancer, skin cancer, small cell lung cancer, non-small cell lung cancer, mesothelioma, gastrointestinal cancer, pancreatic cancer, glioblastoma, vulva cancer, cervical cancer, endometrial carcinoma, ovarian cancer, liver cancer, hepatoma, bladder cancer, kidney cancer, salivary gland carcinoma, thyroid cancer and various head and neck cancers.

122. (canceled)

123. A pharmaceutical compositions comprising compounds illustrated with Formula I, Formula II, Formula III, Formula IV, Formula V, Formula VI, Formula VII, Formula VIII according to claim 1.

124. A pharmaceutical composition according to claim 123, comprising at least one other active agent selected from known active agents that have antiparasitic, antibacterial, antiviral, antineoplastic and/or cytotoxic and/or antimetastatic effects or from combinations comprising two or three of said agents in addition to the compounds illustrated with Formula I, Formula II, Formula III, Formula IV, Formula V, Formula VI, Formula VII, Formula VIII.

125-130. (canceled)
